(19) United States
(12) Patent Application Publication
(10) Pub. No.: US 2021/0353827 A1
(43) Pub. Date: Nov. 18, 2021

(54) MATERIAL FOR BONE IMPLANTS AND METHOD OF PRODUCING SAME

(71) Applicant: stimOS GmbH, Konstanz (DE)

(72) Inventors: HELMUT COELFEN, KONSTANZ-DINGELSDORF (DE); LIANGFEI TIAN, KONSTANZ (DE); JENNIFER KNAUS, KONSTANZ (DE)

(21) Appl. No.: 17/391,736

(22) Filed: Aug. 2, 2021

Related U.S. Application Data
(62) Division of application No. 15/553,555, filed on Aug. 24, 2017.

(30) Foreign Application Priority Data
Feb. 24, 2015 (DE) 10 2015 002 398.5

(51) Int. Cl. A61L 27/34; A61L 27/32; A61L 27/46; A61L 27/12; A61L 27/18; A61L 27/22; A61L 27/24; A61L 27/54

(52) U.S. Cl. CPC A61L 27/34; A61L 27/32; A61L 27/46; A61L 27/12; A61L 27/18; A61L 2300/112; A61L 27/24; A61L 27/54; A61L 2420/04; A61L 2420/06; A61L 2430/02; A61L 27/222

(57) ABSTRACT

The present invention relates to a material for bone implants, comprising: a surface of oxidic ceramic materials, titanium or polyether ether ketone (PEEK) or other polymer or composite materials, a matrix of collagen or gelatin, which is covalently bound to said surface, and calcium phosphate embedded into said matrix. The present invention further relates to a method for producing the material according to the invention, to bone implants comprising the material according to the invention, and to its use as a bone implant material.

### CROSS-REFERENCE TO RELATED APPLICATIONS

This application is a divisional of co-pending U.S. patent application Ser. No. 15/553,555, filed Aug. 24, 2017, which is a National Phase filing under 35 U.S.C. § 371 of International Patent Application No. PCT/EP2016/000174, filed Feb. 3, 2016, which is based upon and claims the priority of German Patent Application No. DE 10 2015 002 398.5, filed Feb. 24, 2015, each of which is incorporated herein by reference in its entirety.

### FIELD OF THE DISCLOSURE

The present invention relates to a material for bone implants, comprising a surface of oxidic ceramic materials, titanium or polyether ether ketone (PEEK), or other polymeric or composite materials, a matrix of collagen or gelatin, which is covalently bound to said surface, and calcium phosphate embedded into said matrix. The present invention further relates to a method for producing the material according to the invention, to bone implants comprising the material according to the invention, and to the use of same as a bone implant material.

### BACKGROUND OF THE DISCLOSURE

In recent years, the number of patients suffering from bone damage and/or needing a bone implant has been on an upward trend. This trend stresses the need to research high-quality, stable and functional bone replacement materials.

The requirements for high-quality and functional bone implants are varied, and it is difficult to satisfy all requirements with one material. Here, the functionality of an implant material is difficult to predict as the natural process of bone wound healing and implant healing is very complex and not yet fully understood in part.

Wound healing of hard or soft tissues after surgery, such as a bone implantation, includes many cellular and extracellular events. The healing process at the contact surface between the bone implant and the bone includes four steps, which take place in a partially overlapping manner. These include inflammatory reactions, the formation of soft callus, followed by the formation of hard callus, and finally the remodeling phase.

After surgery, proteins and other molecules of the blood and lymph initially adsorb on the surface of the implant. If vascularized tissue is inflicted a wound, this will not only lead to an inflammatory reaction, but also to an activation of a variety of other body's own protection systems, such as the extrinsic and intrinsic coagulation system, the complement system, the fibrinolytic system, and the kinin system. These are followed by two sequentially occurring phases that can also take place in an overlapping manner, namely acute and chronic inflammatory reaction. The blood coagulates and forms a blood clot, which is composed of fibrin as a main component. At the same time, cytokines and other growth factors are released to recruit white blood cells to the wound. Here, in the acute inflammatory response, neutrophils and mononuclear cells such as monocytes are recruited. Mononuclear cells differentiate into macrophages and deposit on the surface of the implant. In normal wound healing, macrophages are responsible for cleaning the wound from bacteria, cell debris, and other contaminants via phagocytosis. Here, the implant material is also perceived as a foreign object by the body. However, since the implant is much larger than the macrophages, they cannot phagocytize the material. These events finally lead to the phase of chronic inflammation at the material/tissue boundary. Here, the macrophages merge and form polynuclear giant cells to enclose the foreign object. Macrophages also provide for the recruitment of other cells, such as fibroblasts, which deposit fibrous tissue on the surface of the implant.

After the inflammation phase, a soft callus forms. It consists of bone precursor cells and fibroblasts, which are in a disordered matrix of non-collagenous proteins and collagen. This matrix is gradually formed by said cells as a first reaction, and is structurally similar to the woven bone. The soft callus is finally remodeled gradually from osteoblasts into an ordered lamellar bone structure. In doing so, osteoblasts secrete type I collagen, calcium phosphate, and calcium carbonate with an arbitrary, random orientation. This remodeling phase overlaps with the formation of the hard callus. This takes place by resorption of the disordered bone structures by osteoclasts and subsequent formation of ordered bone structures by osteoblasts.

Primarily, an implant material should be non-toxic and also do not cause inflammatory, immune or other negative or adverse reactions in vivo. If individual particles come loose from the implant, they must also not cause any of the aforementioned reactions and should further be degradable or secretable in the body to avoid permanent accumulation and deposition in the body or aseptic endoprosthesis loosening. Potential bone materials should therefore have reliable strength, high resistance, high abrasion resistance, corrosion resistance, and a stiffness similar to the bone. The latter plays an important role particularly in the context of so-called “stress-shielding”. The bone is a dynamic system that is resorbed or formed depending on mechanical load. Now, if a bone implant material having a higher stiffness compared to the bone substance is used, it will take a large part of the mechanical load, whereby the surrounding bone is resorbed little by little.

Another criterion is the so-called biocompatibility. A potential implant material should be either bioinert or bioactive if possible. A bioinert material does not cause any chemical or biological reactions in the body. A bioactive material, however, promotes rapid growth of the implant into the surrounding tissue and thus ensures a rapid and long-lasting fixation of the implant in the body. This effect is the so-called “osseointegration.” This ability of a biomaterial to promote cell adhesion and migration is crucial for the early stages of wound healing and the later stages of bone regeneration, and depends greatly on the initial contact between the cells and the implant material.

In daily clinical use, autologous bone implants are the “gold standard”. However, the use of autologous bone material of the patient has some drawbacks that limit the application of this approach. For larger bone defects, much bone material is required, which is often not available. A further disadvantage is that a second surgical procedure is necessary to obtain autologous bone material. Therefore, in recent years, a large field dealing with the research of new bone implant materials or the improvement of existing bone implant materials has emerged. There is a wide range of different semi- or fully synthetic materials, such as ceramics, bone cements, polymers, metals, or composite materials, which in some cases are already used in everyday medicine. Each of these materials has beneficial properties, but there is still a great potential for improvement for all. An inherent problem of the ceramics and bone cements, for example, is their brittleness, which leads to mechanical failure of the implant and sometimes even to problems in the biological degradation of the fragments of the implant, which can ultimately lead to aseptic prosthesis loosening. An inherent problem with metal-based materials is their radiopacity. This is disadvantageous in the field of medical imaging diagnostics. Their high rigidity, which can lead to the above-described problems, is a disadvantage as well. Furthermore, metals cannot be referred to as biomimetic materials due to their chemical nature. Also, polymer-based materials, such as polyethylene, polystyrene, or polyether ether ketone (PEEK) have been proposed.

In the field of surface modifications of bone implants, coatings with various calcium phosphates, such as hydroxyapatite or tricalcium phosphate, are widely used. The exact chemical structure of biological apatite is very complex and may generally be regarded as a calcium-deficient hydroxyapatite substituted non-stoichiometrically with carbonate ions. Stoichiometrically pure hydroxyapatite is a form of the apatite with the chemical composition Ca₅(PO₄)₃(OH) and is usually indicated as Ca₁₀(PO₄)₆(OH)₂ in order to illustrate that the primitive cell includes two units. The natural biomineralization is a complex, multi-stage process with a mechanism not yet fully understood. The bone consists of the mineral phase on the other hand, and on the other hand also of 30 to 40% of a protein matrix, which forms the bone matrix. The organic bone matrix in turn consists of several components. 95% consist of collagen. The remaining 5% of the organic matrix mainly consist of proteoglycans and other adhesive glycoproteins.

The collagen of the bone of higher vertebrates consists mainly of type I collagen. Collagen belongs to a group of proteins that constitutes up to 25 to 30% of the total amount of protein of the human body. To date, 28 different types of collagen have been identified in vertebrates, wherein type I occurs most commonly with a frequency of 90% of the total amount of collagen. Type I collagen has the ability to arrange themselves into fibrils. Typically, collagen monomer units arrange themselves in a uniform right-handed triple helix, which usually consists of two identical polypeptide chains (α₁), which in turn are made up of about 334 repeat units of a (Gly-X—Y) sequence, and a further chain (α₂), which slightly varies in its chemical composition. Here, the amino acid proline is often the X position and hydroxyproline is very often the Y position of the repeat unit.

The exact amino acid composition varies with the type and origin of the collagen. Each alpha chain winds in a left-handed helix with about three amino acids per turn, wherein the chain is stabilized via hydrogen bonds. By lateral aggregation, the triple helices can form stable fibers, such as occur in tendons. Here, the monomer units are slightly offset and separated from each other by a gap of about 64 to 67 nm. By aldol condensation between hydroxylysine residues there occurs covalent crosslinking of the fibrils, which provides additional stability, tear and tensile strength. The collagen monomer units are about 300 nm in length and 1.5 nm in diameter. The structural design of collagen fibers is shown in FIG. 1.

A change in just one amino acid of the characteristic repeat unit has great impact on the stability of the entire structure. This can be clearly seen in the disease osteogenesis imperfecta, in which a glycine residue is replaced with other amino acids. The collagen chains thus lose the ability to form stable fibers, which ultimately results in very brittle bones of the patient. Collagen can be isolated from tissues such as calfskin or ligaments and tendons of rat tails.

Collagen as part of the extracellular matrix also contains cellular recognition sequences in its amino acid sequence, which serve for the cellular adhesion of various cell types. An example of such a sequence is the so-called arginylglycylaspartic acid (RGD) motif. Except in collagen, it can be found in fibronectins, vitronectins, fibrinogens, and laminins. In native, fibrillar collagen, this naturally occurring sequence is concealed by the usually triple-helical structure of the collagen. By denaturing the collagen helices, however, these RGD sequences can be exposed and are accessible to the cells. It is presumed that by such a release of the sequences in an injury, wound healing is to be promoted by improved adhesion of the responsible cell types.

In biomineralization of native collagen, a typical sequence may be observed, wherein the mineralization is apparently controlled by other extracellular matrix proteins. These are bound in parallel strands along the collagen fiber. The proteins seem to stabilize the amorphous precursors of the calcium phosphate (amorphous calcium orthophosphate, ACP) and control the subsequent nucleation and growth of the apatite in the gap zone between the collagen monomers. During bone biomineralization, particles primarily in the nanometer range are formed, which then grow rapidly in length and merge with adjacent nanoparticles to finally form needle-shaped structures. These growth processes cause a partial breaking of the bonds between the individual fibers. Finally, the needle-shaped structures merge with adjacent crystals and form plate-shaped or lamellar structures, which grow in the [0 0 1] direction along the c-axis of the collagen fiber. Hence, they have a uniaxial orientation and are also coherently formed into parallel stacks along the ab plane.

For the process of bone mineralization, three compounds are often discussed as intermediate phases. The above-mentioned amorphous calcium phosphate (ACP), octacalcium phosphate (OCP), and dicalcium phosphate dihydrate (DCPD, the mineral brushite) synthetic ACP is also often an intermediate in the precipitation of calcium phosphate from aqueous solution, wherein its chemical composition is highly dependent on the precipitation conditions. The structure of ACP has not yet clearly been defined and appears amorphous in an X-ray diffractogram. On the basis of X-ray absorption spectroscopic measurements, it is also discussed that ACP has an apatitic microstructure with such small domain sizes that it appears X-ray amorphous.

Stoichiometrically pure synthetic OCP has the chemical composition Ca₈(HPO₄)₂(PO₄)₄.5H₂O) and also frequently occurs as a metastable intermediate in the precipitation of calcium phosphate from aqueous solution. From a structural point of view, it is composed of apatite-like layers separated by hydrated layers. Stoichiometrically pure synthetic DCPD has the chemical composition CaHPO₄.2H₂O and as a metastable intermediate can also be crystallized in the precipitation of calcium phosphate from aqueous solution. At temperatures above 80° C. it can easily be converted to dicalcium phosphate anhydrate by dehydration.

In the prior art, bone implant materials have been coated with hydroxyapatite or tricalcium phosphate by various chemical or physical processes. These calcium phosphates are usually produced by simple chemical process by means of precipitation from aqueous solutions. Application onto the surface of the implant material is performed either directly from the solution onto the surface or by means of physical methods, such as “electrospray deposition”. However, in coating of materials with apatite, both the low adhesion of the calcium phosphates on the implant and their limited cohesion within the individual calcium phosphate layers is disadvantageous. These methods were intended to generate a structure, on the surface, which was as similar to the bone as possible in order to promote healing of the material into the bone. However, this ignores the fact that the bone is a highly hierarchical composite material itself, which consists of a matrix and a mineral phase.

Some methods for coating implant materials with collagen were examined for their in vivo functionality. Here, collagen-coated titanium implants, such as screws or nails, were often analyzed in different in vivo systems. Positive effects regarding growing of the material into the surrounding bone and the bone regeneration were reported here. However, there are contradictory results regarding collagen-coated titanium implants in the prior art. For example, improved osseointegration of collagen-coated porous titanium cylinders implanted into the diaphysis of tibial shafts was not found.

Moreover, the prior art reports of methods for covalent or non-covalent immobilization of various proteins of the extracellular matrix, such as fibronectin or short peptides, on surfaces. They partly showed positive effects on cell adhesion and proliferation in in vitro test systems.

Frequently, collagen is replaced by its denatured form gelatin for cost and handling reasons. Gelatin is usually produced by physical and chemical degradation or thermal denaturation of native collagen. In contrast to native collagen, gelatin is soluble in water at a physiological pH value and melts at a sol-gel transition temperature of 25 to 30° C. Transparent gels form after cooling. The prior art also reports the non-covalent application of gelatin on surfaces of arterial implant materials.

### BRIEF SUMMARY OF THE INVENTION

On this basis, it is the object of the present invention to provide a material for bone implants having bone-like structures, which include both the protein and the mineral phases of natural bone. In addition, a corresponding production method is to be provided.

This object is solved by the embodiments characterized in the claims.

### BRIEF DESCRIPTION OF THE DRAWINGS

The construction and method of operation of the invention, however, together with additional objects and advantages thereof will be best understood from the following description of specific embodiments when read in connection with the accompanying drawings.

The figures show:

FIG. 1: Structural design of collagen fibers.

FIG. 2: Structural formula of polyether ether ketone (PEEK).

FIG. 3: ATR-IR spectrum of the unmodified PEEK.

FIG. 4: Overview spectrum of XPS measurement of the unmodified PEEK.

FIGS. 5A and 5B: Fine spectra of the C1S signal (FIG. A) and the O1S signal (FIG. B) of the XPS measurement of the unmodified PEEK.

FIG. 6: 1H-NMR spectrum of the unmodified PEEK.

FIGS. 7A-7D: Result of the atomic-force microscopic examination of the unmodified PEEK. FIG. 7A: overview of a 50 μm×50 μm surface; FIG. 7B: height profile for the image of FIG. 7A; FIG. 7C: overview of a 4 μm×4 μm surface; FIG. 7D: height profile for the image of FIG. 7C.

FIG. 8: Reaction scheme of the reduction of the PEEK.

FIG. 9: 1H-NMR spectrum of the PEEK-OH.

FIG. 10: ATR-IR spectrum of the PEEK-OH.

FIG. 11: Overview spectrum of XPS measurement of the PEEK-OH.

FIGS. 12A and 12B: Fine spectra of C1S signal (FIG. 12A) and the O1S signal (FIG. 12B) of the XPS measurement of the PEEK-OH.

FIG. 13: Theoretical values of the C1S and O1S components and their mapping to the chemical structure of the PEEK-OH.

FIGS. 14A and 14B: Results of the atomic-force microscopic examination of the PEEK-OH. FIG. 14A: surface representation, FIG. 14B: height profile.

FIG. 15: Reaction scheme of the coupling of the succinic acid linker.

FIG. 16: ATR-IR spectrum of the PEEK-COOH.

FIG. 17: 1H-NMR spectrum of the PEEK-COOH.

FIG. 18: Reaction scheme of the coupling of the maleimide to PEEK-OH.

FIG. 19: ATR-IR spectrum of the PEEK-OH compared to PEEK-maleimide.

FIG. 20: Reaction scheme of the coupling of hexamethylene diisocyanate to PEEK-OH.

FIG. 21: ATR-IR spectra of the PEEK-OH compared to PEEK-NCO.

FIG. 22: Overview spectrum of XPS analysis of the PEEK-NCO.

FIGS. 23A-23C: Fine spectra of the XPS measurement of the PEEK NCO of O1S (FIG. 23A), C1S (FIG. 23B), and N1S transition (FIG. 23C).

FIG. 24: Reaction scheme of the coupling of gelatin to PEEK-COOH.

FIG. 25: Result of the REM and subsequent EDX examinations as to nitrogen on the surface of the PEEK-gelatin film.

FIGS. 26A-26C: Result of the REM examination of an edge face of the surface of the PEEK-gelatin film (FIG. 26A) overview, (FIG. 26B) close-up of the surface, (FIG. 26C) unmodified PEEK surface as a reference.

FIG. 27: Reaction scheme of the coupling of gelatin via an isocyanate linker.

FIG. 28: Result of the REM examination of the surface of the PEEK gelatin film via isocyanate linker.

FIG. 29: Result of the REM examination of the surface of the PEEK gelatin film via maleimide linker.

FIG. 30: Results of the REM examination of the surface of mineralized PEEK-HAp film.

FIGS. 31A-31F: REM image (FIG. 31A) of the mineralized sheet cut with FIB and its complementary TEM image (FIG. 31B). FIG. 31C, FIG. 31D: TEM image of the mineralized boundary surface and electron diffraction images at the TEM image sections shown. FIG. 31E: TEM image of an ultramicrotomy cut of the mineralized sheet. FIG. 31F: electron diffraction image of the mineral layer at the boundary surface.

FIG. 32: Results of the proliferation test of NHDF on PEEK, PEEK gelatin and PEEK-HAp.

FIG. 33: MTT viability test of NHDF on PEEK, PEEK gelatin and PEEK-HAp.

FIG. 34: Results of the adhesion test of NHDF on PEEK, PEEK gelatin and PEEK-HAp.

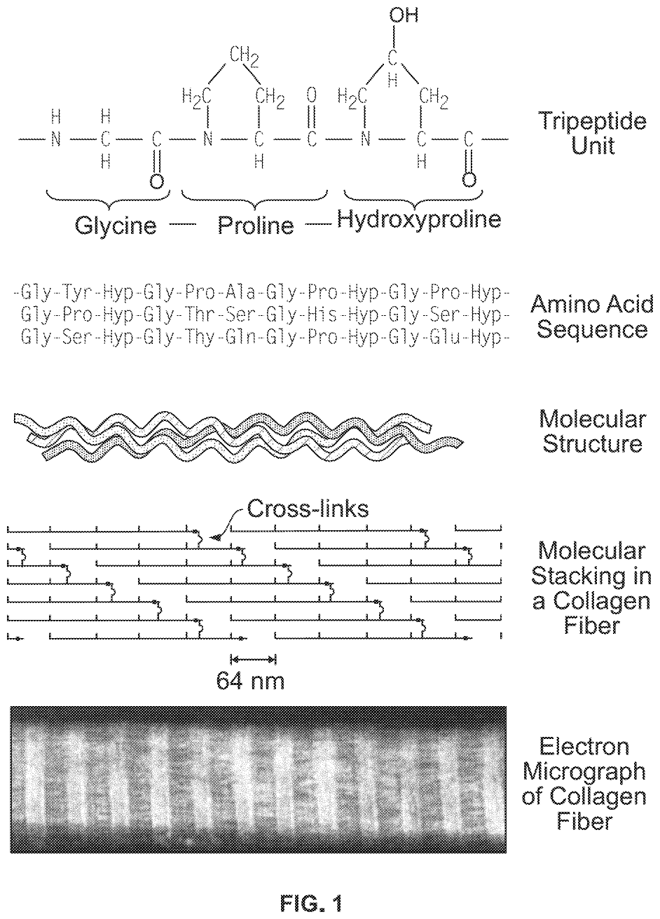

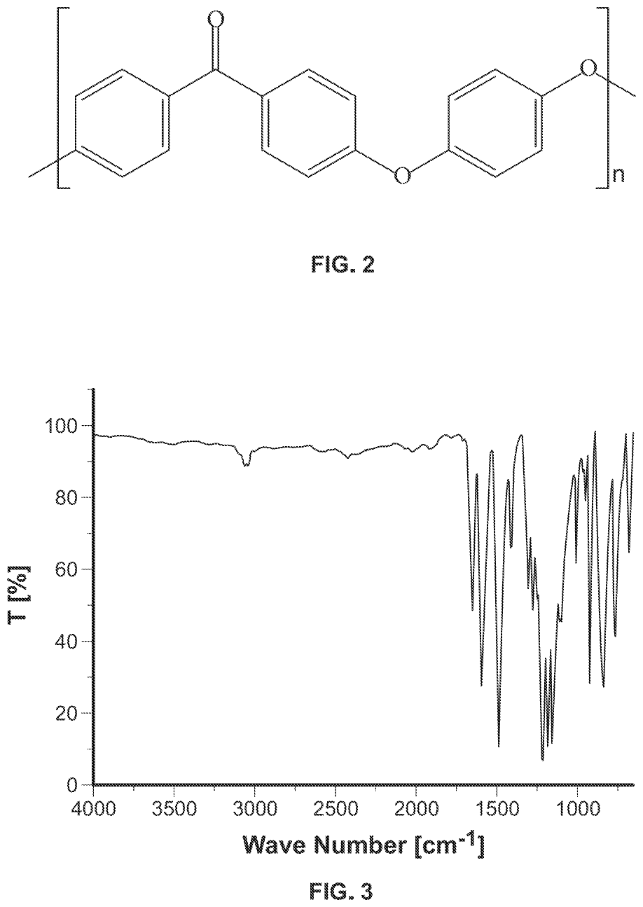

### DETAILED DESCRIPTION OF THE INVENTION

In particular, a subject matter of the present invention relates to a material for bone implants, comprising:
(a) a surface comprising a material selected from the group consisting of oxidic ceramic materials, titanium, polymer materials, and composite materials,
(b) a matrix covalently bound to this surface, comprising collagen and/or gelatin, and
(c) calcium phosphate embedded into this matrix.

The terms “material for bone implants” and “bone implant material” are used synonymously herein. The inventive material for bone implants has bioactive properties. As used herein, the term “bioactive” refers to the property of the inventive material for bone implants of permitting rapid growth into the surrounding tissue and ensuring a rapid and long-lasting fixation of the implant in the body. This property results solely from the technical features defined in the above subitems (a) to (c).

The inventive material for bone implants is applied to solid materials or bodies, which are used as a bone implant. These bodies may have any desired or required three-dimensional shape. Preferably, the entire surface of the inventive material for bone implants comprises the material defined in the above subitem (a) or is composed thereof. Suitable materials to which the inventive material can be applied may be selected from ceramic materials, metals, polymers, composite materials or combinations thereof well-known in the prior art.

The surface of the inventive material for bone implants comprises a material selected from the group consisting of oxidic ceramic materials, titanium, polymer materials, in particular polyether ether ketone (PEEK), and composite materials, or is composed thereof. Suitable oxidic ceramic materials, polymer materials, and composite materials are known in the prior art. In a preferred embodiment, the material is PEEK. This material is mechanically very similar to native bone material and well thus suitable as a bone implant material.

Specifically, the inventive material for bone implants comprises a matrix covalently bound to the surface, said matrix comprising collagen, preferably type I collagen and/or gelatin. In certain embodiments, this matrix consists of collagen, preferably type I collagen and/or gelatin. Gelatin is a denatured form of collagen and is more cost-effective and easier to handle compared thereto. Therefore, the use of gelatin as the matrix material is preferred. Methods for covalently bonding a matrix of collagen and/or gelatin to a surface will be described hereinafter. The covalently bound matrix typically has a layer thickness of 100 to 150 nm, but may also be thicker or thinner. In particular, the covalently bound matrix may have a layer thickness of 1 nm to 10 nm, preferably from 10 nm to 1 nm, more preferably from 20 nm to 500 nm, more preferably from 30 nm to 300 nm, more preferably from 50 nm to 200 nm, and most preferably from 100 to 150 nm. Further, the covalently bound matrix preferably covers the entire surface of the inventive material for bone implants.

Finally, the inventive material for bone implants comprises calcium phosphate embedded into said matrix, preferably calcium orthophosphate in all mineral forms, particularly preferably selected from the group consisting of amorphous calcium orthophosphate (ACP), dicalcium phosphate dihydrate (DCPD; brushite), octacalcium phosphate, and hydroxyapatite, also with partial fluoride, chloride or carbonate substitution, wherein ACP, hydroxyapatite, and octacalcium phosphate are particularly preferred. Methods for embedding said calcium phosphates into a corresponding matrix will be described hereinafter.

In a preferred embodiment, the inventive material for bone implants comprises PEEK or is composed thereof, wherein the collagen and/or gelatin of the covalently bound matrix are bound to the PEEK via a linker selected from the group consisting of a dicarboxylic acid linker, a maleimide linker, and a hexamethylene diisocyanate linker. Corresponding linkers are known in the prior art. Methods for covalently bonding collagen and/or gelatin to PEEK will be described hereinafter.

In other embodiments, the inventive material for bone implants comprises oxidic ceramic materials, titanium, polymer materials, or composite material, or is composed thereof, wherein the collagen and/or gelatin of the covalently bound matrix are bound via a silane linker. Suitable silane linker and corresponding methods for bonding collagen and/or gelatin are known in the prior art.

In a particularly preferred embodiment, the present invention relates to a material for bone implants, comprising:
(a) a surface consisting of PEEK,
(b) a matrix covalently bound to this surface, consisting of gelatin, and
(c) hydroxyapatite embedded into this matrix.

Another subject matter of the present invention relates to a method for producing an inventive material for bone implants, comprising the steps of:
(a) providing a surface comprising a material selected from the group consisting of oxidic ceramic materials, titanium, polymer materials, and composite materials,
(b) covalently coupling a matrix, comprising collagen and/or gelatin, to this surface and
(c) mineralizing the matrix with calcium phosphate.

To this subject matter of the present invention, all relevant definitions and preferred embodiments described above for the inventive material for bone implants apply analogously.

Methods for covalently coupling a matrix comprising collagen and/or gelatin to a surface according to step (b) of the inventive method are not particularly limited and are known in the part art.

In a preferred embodiment, the surface comprises PEEK or consists thereof, and step (b) of the inventive method comprises the steps of:
(b1) activating the surface by reducing the keto group of the PEEK to form a hydroxyl group,
(b2) covalently coupling a linker molecule selected from the group consisting of a dicarboxylic acid linker, a maleimide linker, and a hexamethylene diisocyanate linker to this activated surface, and (b3) covalently coupling the collagen and/or gelatin to the linker molecule.

Methods for reducing the keto group of PEEK to form a hydroxyl group are not particularly limited and, for example, comprise incubating the surface with a solution of sodium borohydride and dimethyl sulfoxide or a solution of lithium aluminum hydride in organic solvents.

Methods for coupling linker molecules to a correspondingly activated PEEK surface also are not particularly limited. In cases where the linker molecule is a dicarboxylic acid linker, the methods comprise e.g. incubating the surface with a solution containing the corresponding dicarboxylic acid, N,N′-dicyclohexylcarbodiimide (DCC) and 4-dimethylaminopyridine (DMAP) in tetrahydrofuran (THF). In cases where the linker molecule is a maleimide linker, the methods e.g. comprise incubating the surface with a solution containing triphenylphosphine, diethyl azodicarboxylate, and maleimide in THF. In cases where the linker molecule is a hexamethylene diisocyanate linker, the methods e.g. comprise incubating the surface in an inert gas atmosphere and dry reaction conditions with a solution containing hexamethylene diisocyanate and catalytic amounts of 1,4-diazabicyclo[2.2.2]octane (DABCO) in toluene.

Methods for coupling collagen and/or gelatin to corresponding linker molecules are not particularly limited and are known in the prior art. If the linker molecule is a dicarboxylic acid for example, step (b3) of the method according to the invention comprises incubating the surface with a 1-ethyl-3-(3-dimethylaminopropyl)carbodiimide (EDC)/N-hydroxysuccinimide (NHS) solution (EDC/NHS solution), and subsequently incubating the surface with a solution containing collagen and/or gelatin. If the linker molecule is a maleimide linker or a hexamethylene diisocyanate linker, step (b3) of the method according to the invention comprises incubating the surface with a solution containing collagen and/or gelatin.

Methods for mineralizing a matrix containing collagen and/or gelatin with calcium phosphates according to step (c) of the method according to the invention are not particularly limited. In the case that ACP is used, they comprise e.g. incubating the surface with a solution comprising calcium chloride, dipotassium phosphate, and a nucleation inhibitor. This nucleation inhibitor is preferably a non-collagenous protein or protein analogue, more preferably poly-aspartic acid and/or fetuin. In the event that hydroxyapatite is used, they comprise e.g. incubating the surface with a solution comprising calcium chloride and dipotassium phosphate.

Another subject matter of the present invention relates to a bone implant comprising a solid material and/or a solid body to which the inventive bone implant material is applied.

To this subject matter of the present invention, all relevant definitions and preferred embodiments described above for the inventive material for bone implants apply analogously.

Another subject matter of the present invention relates to the use of the inventive material for bone implants as a bone implant material.

To this subject matter of the present invention, all relevant definitions and preferred embodiments described above for the inventive material for bone implants apply analogously.

Another subject matter of the present invention relates to the use of the inventive material for bone implants for treating bone damage, for example.

To this subject matter of the present invention as well, all relevant definitions and preferred embodiments described above for the inventive material for bone implants apply analogously.

Another subject matter of the present invention relates to the use of the inventive bone implant for treating bone damage, for example.

To this subject matter of the present invention as well, all relevant definitions and preferred embodiments described above for the inventive material for bone implants apply analogously.

The better the implant surface corresponds to the natural bone, the better implants grow into the human body and are more stably connected to the body (among others, by increased accumulation of body cells). This is the object of the present invention. The chemical composition and especially the surface structure of the coating down to the micro- and even nanometer range is to come as close to the natural bone structure as possible according to the invention. Further, the coating is to be bound covalently to the surface of the implants. The bone implant materials of the invention have a higher biocompatibility, better healing into the natural bone, and an increased mechanical load-bearing capacity.

The surface modification according to the present invention aims at applying bone-like structures to the surface of the bone implant materials in a covalently bound manner, which include both the protein and the mineral phases of natural bone. This is to support healing of the implant into the bone. These structures include a matrix of denatured collagen, gelatin, which is finally mineralized with calcium phosphate. Here, mineralization takes place with the aid of non-collagenous proteins and their analogues, which act as nucleation inhibitors, so that the mineralization occurs in a controlled way and ectopic mineralization is avoided. Such nucleation inhibitors are, for example, poly-aspartic acid or fetuin. Mineralization with octacalcium phosphate or hydroxyapatite takes place by incubating the gelatin matrix in a solution containing calcium ions or phosphate ions. By a slow and controlled addition of a solution of the respectively complementary phosphate ions or calcium ions, octacalcium phosphate and/or hydroxyapatite can be precipitated within the gelatin matrix. Due to the relatively disordered structure of the gelatin, the resulting surface modification has a woven bone-like or callus-like structure. Thus, during healing of the material, the bone cells could build more disordered collagen structures around the material or link the material directly further to the bone. These disordered structures can finally be rebuild into ordered bone structures in the natural remodeling phase of bone healing. Here, however, the cells cannot penetrate up to the direct surface of the implant material due to the covalent bonding of the proteins in the remodeling phase, and thus always remain in a desired matrix of extracellular proteins. The implant material is thus masked for the cells to avoid adverse reactions in the healing of implants. Since the modification only relates to the surface of the implant materials, material properties are not changed.

The basic chemical reactions can be easily adapted for the modification of various materials. For example, metal oxide surfaces can be covalently bound via established silane chemistry. This makes the surface coating of the invention attractive also with respect to oxide ceramics. Furthermore, as metals can be oxidized easily on the surface by plasma treatment, also the common implant materials made of titanium become accessible to the inventive surface modification by silanes via silane chemistry.

The present invention will be explained on the basis of the following non-limiting examples.

### Example 1

### Analysis of Unmodified PEEK Material

The purchased material was PEEK optima by Invibio, Hofheim. These sheets had an amorphous structure and therefore appeared transparent with a white-beige color. An ATR-IR spectrum of the unmodified material can be seen in FIG. 3 and shows a spectrum that matches well with literature results. Typical bands for aromatic polymers such as PEEK are the aromatic stretching vibrations at 1650 cm⁻¹, 1593 cm⁻¹, and 1486⁻¹, as well as the diaryl ether stretching vibration at 1216 cm⁻¹. As PEEK has a relatively strong autofluorescence, it was not possible to carry out a complementary Raman study.

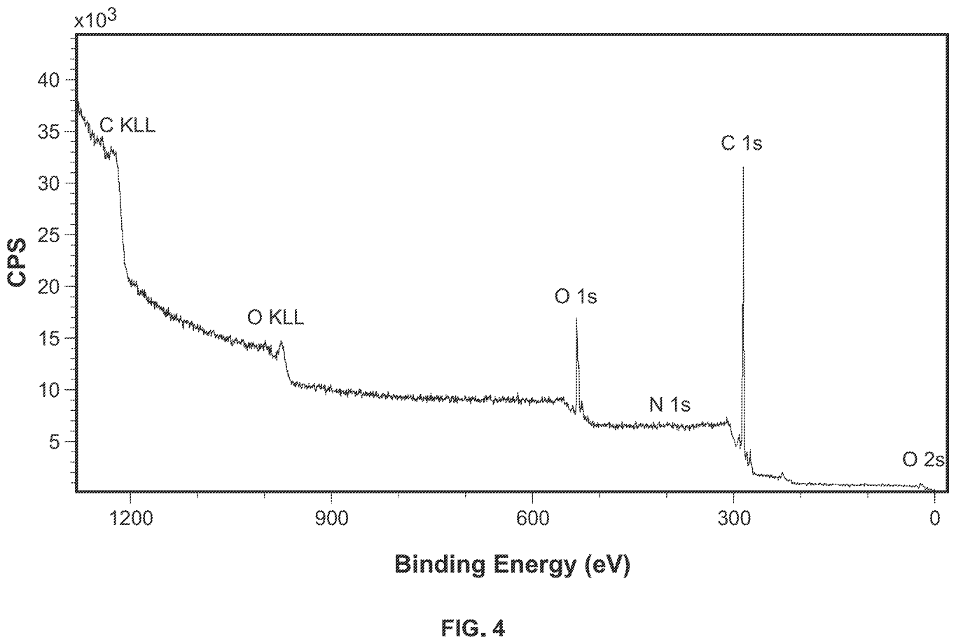

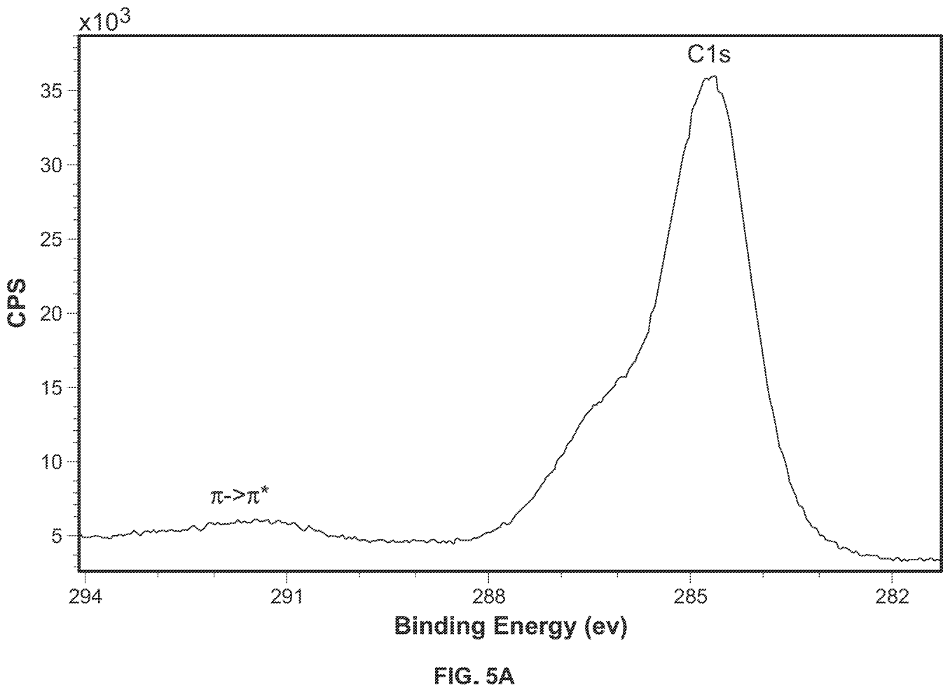

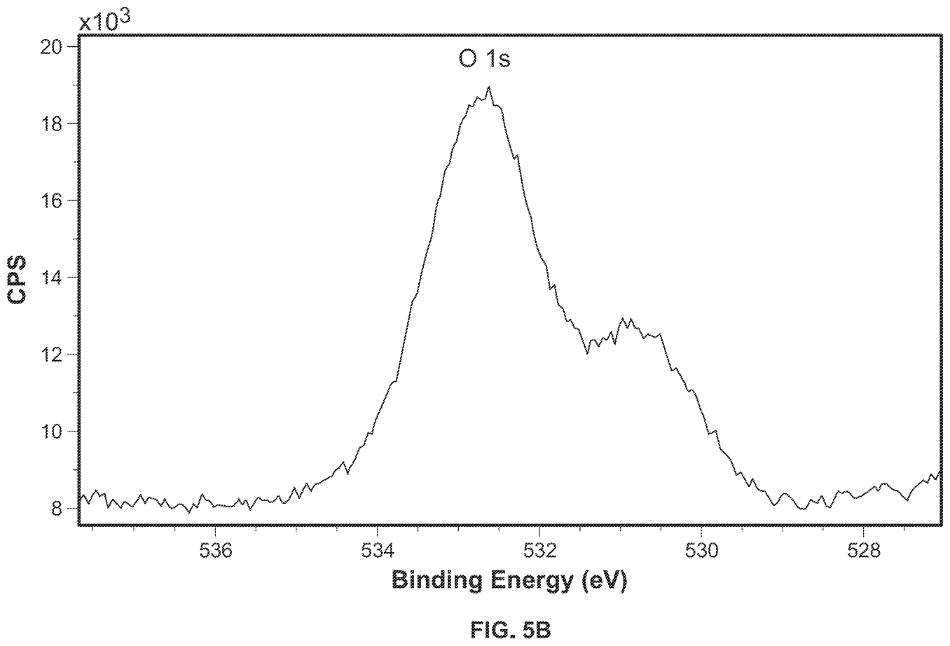

Furthermore, the surface was examined with X-ray photoelectron spectroscopy (XPS). An overview spectrum is shown in FIG. 4, and the results are summarized in Table 1. Since PEEK is a non-conductive material, there was charging phenomena during the measurement, which resulted in charge equilibrium and a concomitant shift of the signals by a few electron volts. This could not be minimized in the instrumental set-up due to the absence of a compensation method. Carbon has three different chemical environments in PEEK, which result in three identifiable and distinguishable bonding energies between the atoms of the C_{1S} transition. The C_{1S} spectrum (FIG. 5A) shows a C—C/C—H component at 284.7 eV, a C—O component at 286.3 eV, a C═O component at 287.1 eV, and a π-transition satellite peak eV at 291.4. They have a relative composition of 75.6%, 17.7%, 3.6%, and 3.1%, respectively. Oxygen has different chemical environments in PEEK, which result in two bonding energies that are close to each other. The O_{1S} fine spectrum (FIG. 5B) shows an O═C component at 531, 1 eV and an O—C component at 533.3 eV with a relative concentration of 26.4% and 73.6%. The total concentration of the carbon and oxygen in PEEK was 74.6% and 24.2%. Herewith, a C:O ratio of 3.03 was calculated. Since the theoretically expected value of a stoichiometrically composed PEEK repeat unit is 6.33, it is believed that water or other oxygen-containing compounds were present on the surface during the measurement. Since the material was not explicitly dried prior to the measurement and was transferred into the measuring chamber under dry conditions, it seems to be possible that humidity adsorbed on the surface of the material. On the other hand, a nitrogen-containing component at the surface of the material of 1.2% was also determined. This could result from the manufacturing process of the material or contaminations during transport.

TABLE 1
Summary of the results of XPS analysis of unmodified PEEK.

| | | | Component | Element |
|---|---|---|---|---|
| | Element/ | BE | Composition | Concentration |
| Assignment | Transition | [eV] | [at. %] | [at. %] |
| Carbon | C 1s | 284.7 | 75.6 | 74.52 |
| | C 1s | 286.3 | 17.7 | |
| | C 1s | 287.1 | 3.6 | |
| | C 1s | 291.4 | 3.1 | |
| Oxygen | O 1s | 531.1 | 26.4 | 24.28 |
| | O 1s | 533.3 | 73.6 | |
| Nitrogen | N 1s | | 100 | 1.2 |

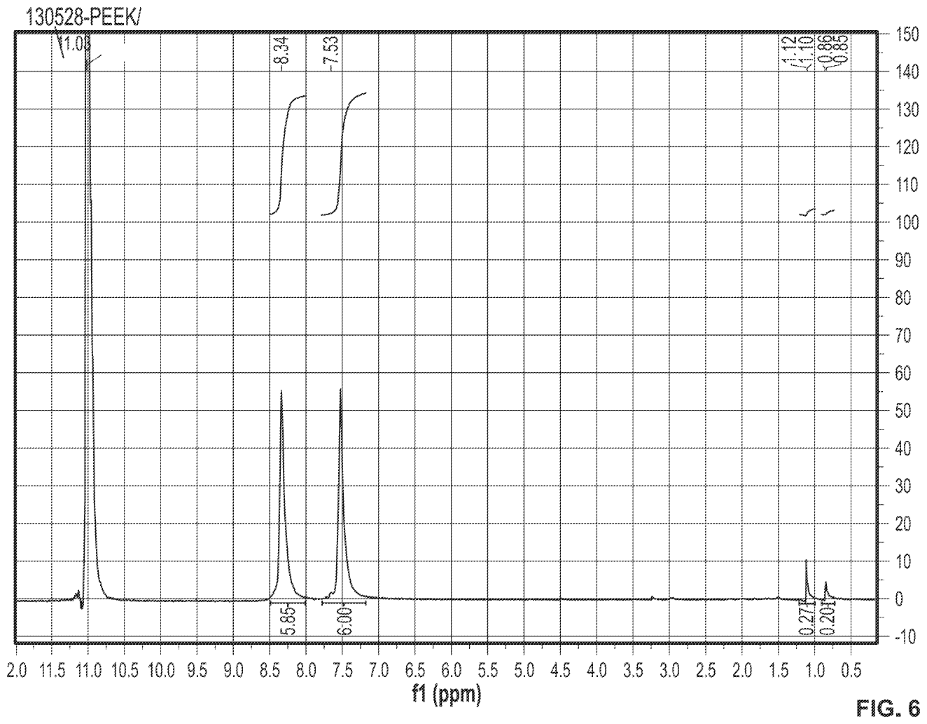

Furthermore, NMR spectroscopic studies with CF.sub.3SO.sub.3D as a solvent were carried out. The ¹H-NMR spectrum is shown in FIG. 6 and has two characteristic peaks at 8.34 ppm and 7.53 ppm, each with the same intensity. These were attributed to the aromatic protons. Moreover, two further signals were measured at 1.11 ppm and 0.85 ppm, each a doublet. They probably result from aliphatic protons. This gives rise to the assumption of contaminations on or in the material, as there should not be aliphatic protons in pure PEEK. An HSQC-NMR experiment showed a bonding of the aliphatic protons to carbon atoms, which appear at a shift of 163.4 ppm and 163.19 ppm.

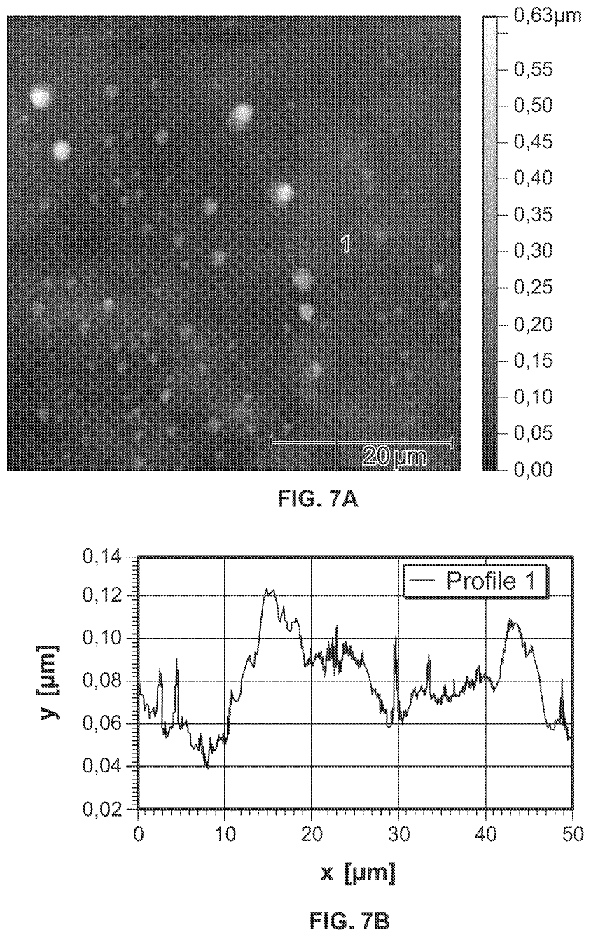

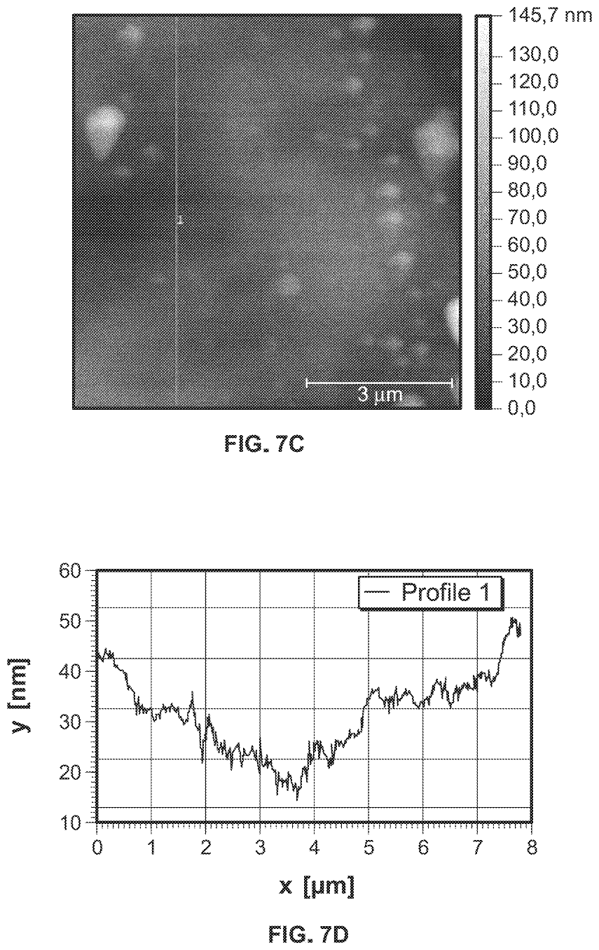

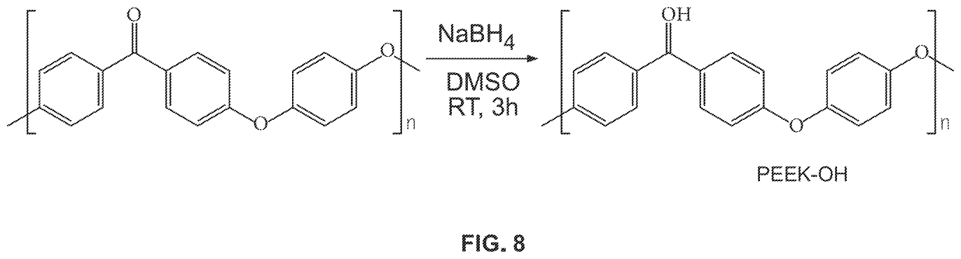

The surface of the material was further characterized using atomic force microscopy (AFM). The results are shown in FIGS. 7A and 7B. The surface has a relatively smooth and uniform structure. It is likely that the sporadic unevenness on the surface result from dust particles in the air, as the atomic force microscope is not in a dust-free environment. The maximum height difference was prepared using an overview image (FIGS. 7B, 7D, Profile 2) and is 433 nm with measured dust particles, and about 17 to 33 nm without them.

An important factor for increasing the biocompatibility is to increase the hydrophilic nature of the surface of the material, as this results in excellent wetting by the blood and body fluid present in the surgical wound of the bone and the related adhesion of osteogenesis-stimulating factors. Thus, a complementary surface characterization was carried out by water contact angle measurements. The contact angle was 89°±1°. This corresponds to low hydrophily or low wettability of the material.

### Example 2

### Wet-Chemical Modifications of PEEK—Reduction to PEEK-OH

In order to increase the biocompatibility of the material, the surface needs to be activated at first in order to chemically bond biopolymers, such as gelatin, afterward.

In the case of the PEEK material, the keto group was at first reduced to a hydroxyl group to serve as an anchor point between the gelatin layer and the material for the subsequent coupling reactions. This was done according to a modification known in the prior art. To this end, a PEEK sheet was immersed in a solution of sodium borohydride in dimethylsulphoxide. The resulting product PEEK-OH will be mentioned hereinafter.

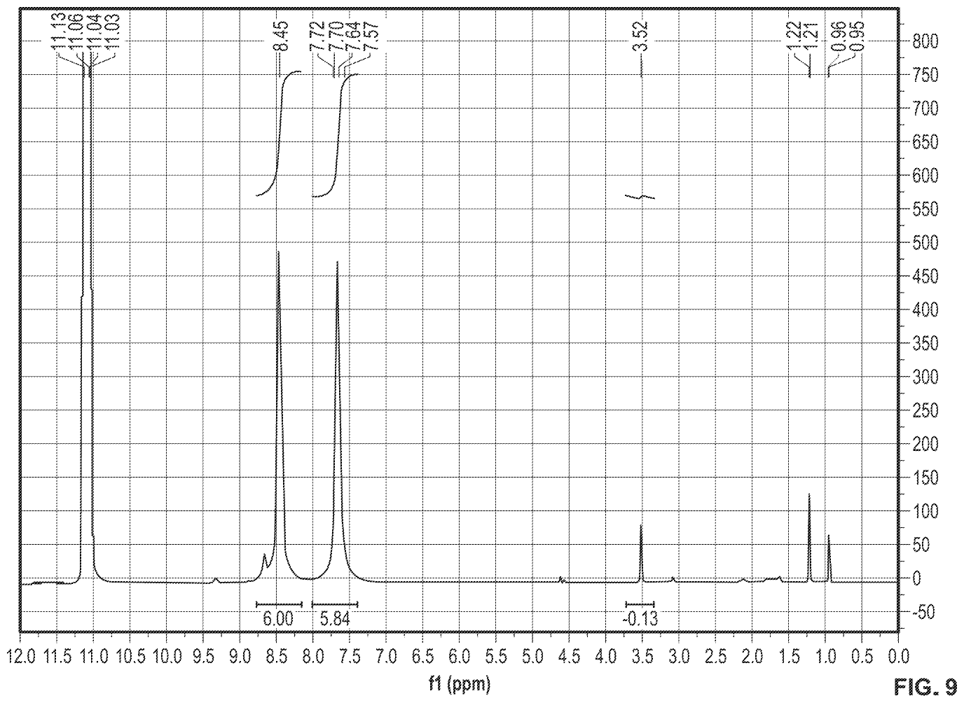

To verify the success of the reaction, NMR spectroscopy has been used (FIG. 9). Since the unmodified material only has aromatic protons, the appearance of non-aromatic protons in a ¹H-NMR experiment to should correlate directly with the hydroxyl modification and thus the success of the reaction.

Also, the aliphatic protons can be distinguished well from the resulting benzhydryl-proton signals and identified by a before/after comparison. The ¹H-NMR spectrum shows a singlet at δ=8.56 ppm, which is typical of a carbocationic species. The existence of this species is hardly surprising due to the use of a very strong acid as a solvent, which might have dehydrated the PEEK-OH. The carbocation is well resonance-stabilized due to its positioning between two aromatic systems, and can be measured this way. Furthermore, a further new signal can be taken from the spectrum at 3.41 ppm, which might correspond to the benzhydryl proton. The fine splitting of the signal is a doublet with a coupling constant of 3.1 Hz. This might result from the ⁴J-coupling of the benzhydryl proton to the aromatic protons. An HSQC-NMR experiment showed a coupling of the carbocationic proton signal to a carbon signal, which appears at a shift of 113.8 ppm. The signal identified as benzhydryl couples to a carbon signal at a shift of 34.5 ppm, which would correspond to a success of the reaction.

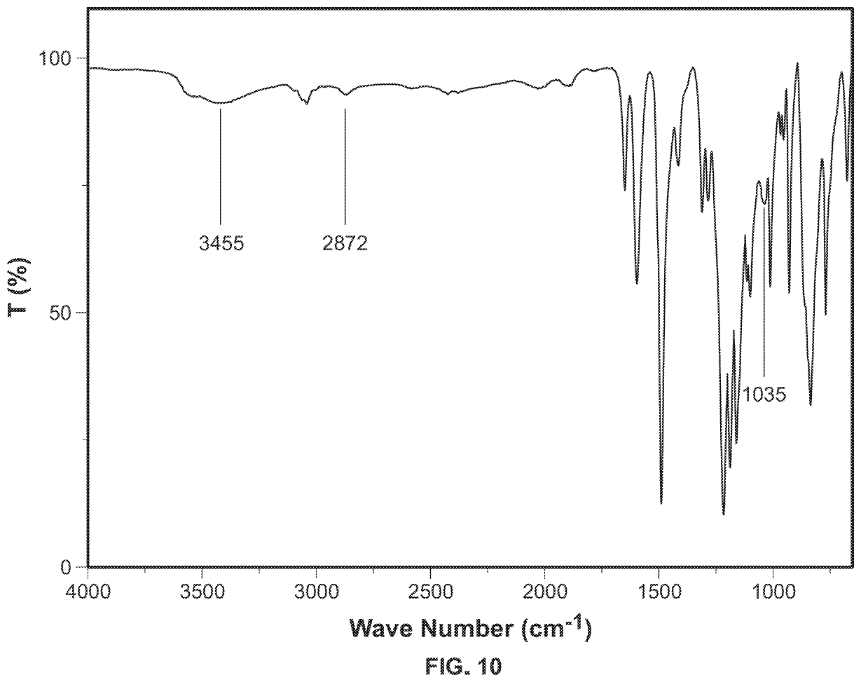

Further, the surface was analyzed by ATR-IR spectroscopy. The spectrum is shown in FIG. 10. In comparison to the unmodified material, the spectrum has a broad band at about 3400 cm⁻¹, which was attributed to an OH stretching vibration, a new band at 2874 cm⁻¹, which was attributed to a C—H stretching vibration, and a new band at 1035 cm⁻¹, which was attributed to a C—O stretching vibration of the alcohol.

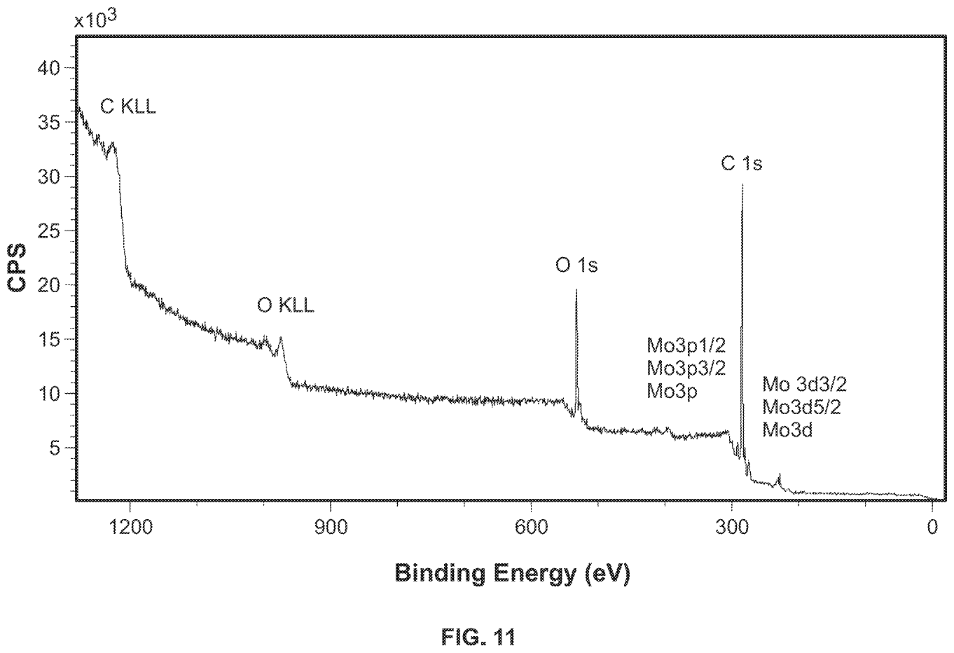

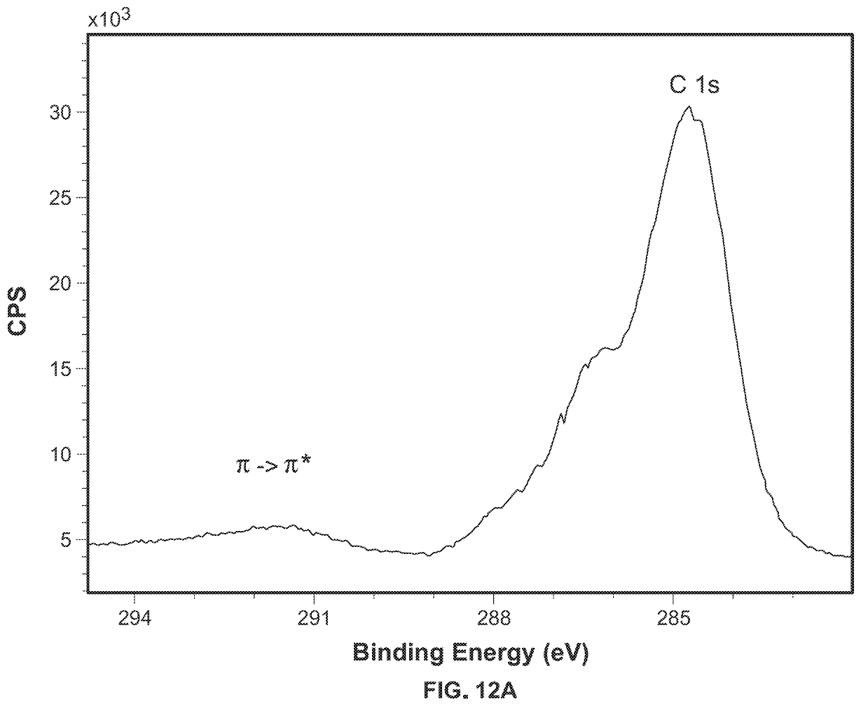

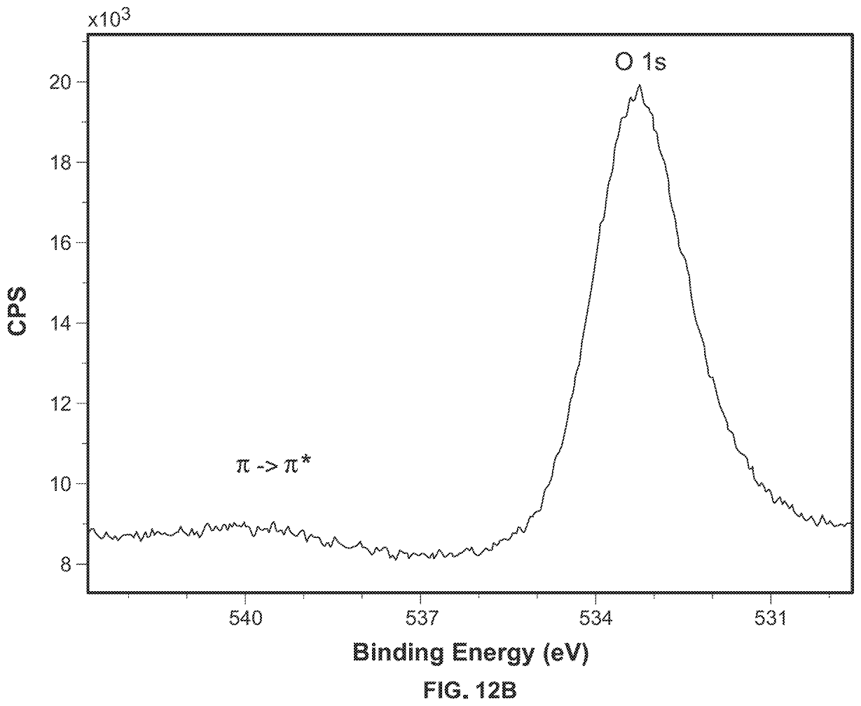

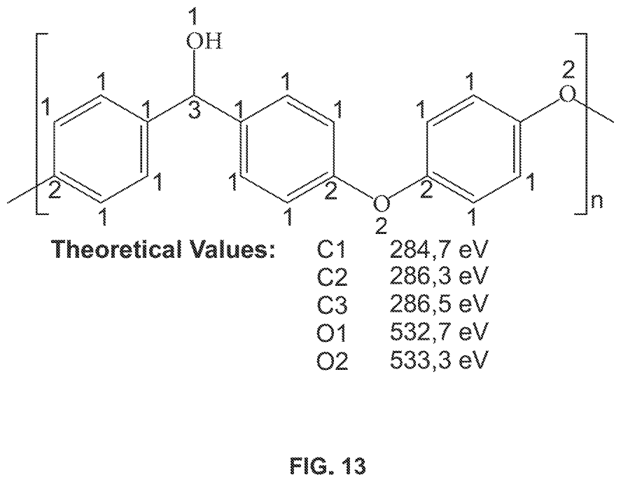

The surface of the material was further analyzed with XPS and compared to the unmodified material. The overview spectrum and the corresponding fine spectra are shown in FIGS. 11 and 12A and 12B. To determine and compensate for surface charging during the measurement, a molybdenum reference was used. The results are summarized in Table 2. The fits were created on the basis of theoretical values of relevant carbon and oxygen bonds of polymers. The C.sub.1S fine spectrum shows a C—C/C—H component at 284.7 eV, a C—O component at 286.3 eV, a C═O component at 286.5 eV, and a 7-transition satellite signal at 291.5 eV. These signals show a relative composition of 63.07%, 32.25%, 2.34%, and 3.32%. Thus, a reduction of the C═O component and a concomitant increase in the C—O component were measured. The O_{1S} fine spectrum shows an O═C component at 531.1 eV, an O—C component at 532.2 eV, an O—H component at 533.3 eV, and a 7-transition satellite signal at 539.8 eV. They have a relative composition of 11.1%, 32.2%, 51.4%, and 5.3%. A C:O rate of 3, 16 resulted from these values. Since the theoretically calculated value for a stoichiometrically composed repeat unit of PEEK is 6.33, it is assumed that not 100% of the keto-groups are reduced to hydroxyl groups or that, in turn, impurities from the above-mentioned sources are present on the surface. Theoretical values of the C_{1S} and O_{1S} components and their assignment to the chemical structure of PEEK-OH are shown in FIG. 13.

TABLE 2
Summary of the XPS analysis of PEEK-OH.

| | | | Component | Element |
|---|---|---|---|---|
| | Element/ | BE | Composition | Concentration |
| Assignment | Transition | [eV] | [at. %] | [at. %] |
| Carbon | C 1s | 284.7 | 63.09 | 68.4 |
| | C 1s | 286.3 | 32.25 | |
| | C 1s | 286.5 | 2.34 | |
| | C 1s | 291.5 | 2.32 | |
| Oxygen | O 1s | 531.1 | 11.1 | 31.6 |
| | O 1s | 532.2 | 32.2 | |
| | O 1s | 533.3 | 51.4 | |
| | O 1s | 539.8 | 5.3 | |

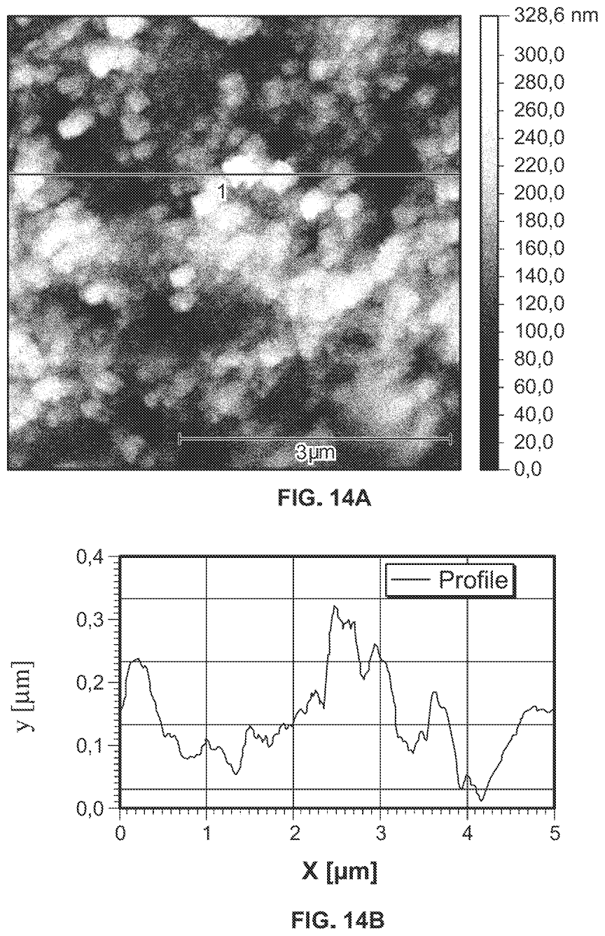

The surface of the PEEK-OH material was further examined with atomic force microscopy. Before the reaction, the maximum height difference was 17 to 33 nm in a 16 μm² analysis area. After the reaction, the maximum height difference was 208 nm (FIGS. 14A and 14B). This relatively large height difference led to further complications for the continued use of this method for the analysis of the material. When gelatin is coupled to the surface in the next step, the height difference and the structure could not be pronounced sufficiently in order to distinguish the gelatin layer from the bulk material. For this reason, further AFM measurements were dispensed with hereinafter.

Measurements of water contact angle showed a decrease of the contact angle from previously 89° of the unmodified material to 77° of the modified material, which means an increase in the hydrophilicity of the surface.

Due to the strong autofluorescence of the PEEK material, no fluorescent dyes were used as indirect evidence and for a possible quantification.

### Example 3

### Coupling of Linker Molecules—PEEK-COOH

In order to ensure covalent binding of the gelatin molecules to the PEEK-OH surface, it has to be modified chemically with appropriate linker molecules. The reactions specified below are possible here. In the case of metal oxide surfaces, silanes would be used as linker molecules.

In order to create a surface with acid groups, a linker molecule was bound to the surface via an ester bond. This should be a dicarboxylic acid, which is interconnected by methylene groups of a variable number. Exemplarily, the reaction was carried out on a succinic acid linker.

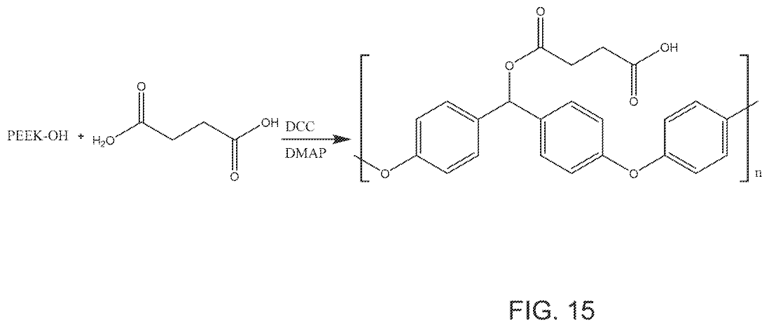

This was realized with the help of N,N′-dicyclohexylcarbodiimide (DCC) and 4-dimethylaminopyridine (DMAP). Typically, during the reaction procedure, two equivalents of succinic acid were initially dissolved in tetrahydrofuran (THF) and cooled with an ice bath. Then one equivalent of DCC and 0.1 equivalent of DMAP were added to the cooled solution. After four hours, the PEEK-OH sheets were added into this solution and stirred for a further four days at room temperature. Thereafter, the sheets were washed three times with THF and three times with acetone. Drying was finally carried out in a vacuum oven at 40° C. and 50 mbar. The reaction scheme of the coupling of the succinic acid linker is shown in FIG. 15.

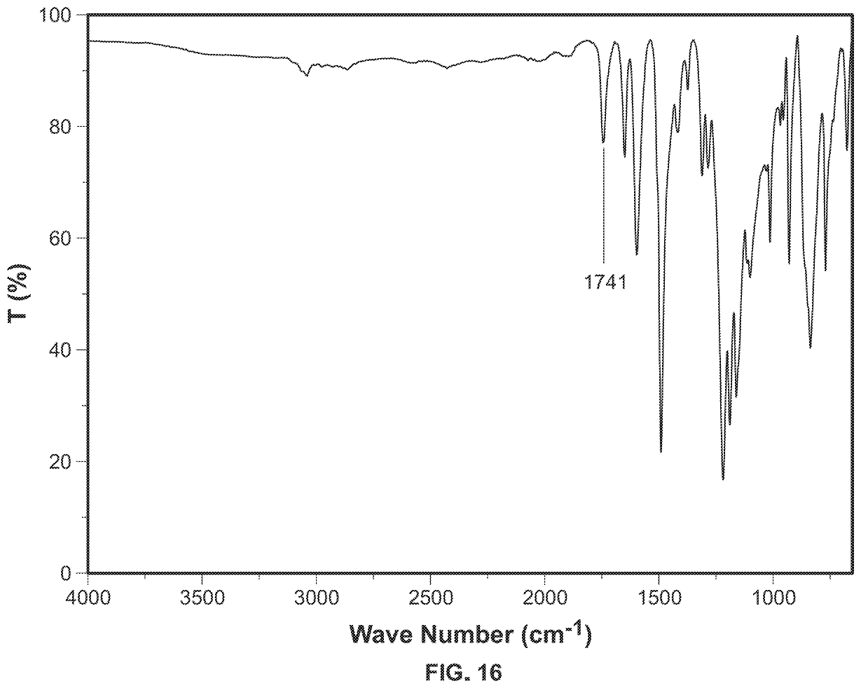

The sheets were examined with ATR-IR spectroscopy (FIG. 16). In the spectra, a new band was identified at 1741 cm⁻¹, which is characteristic of an ester group and thus is indicative of the success of the reaction.

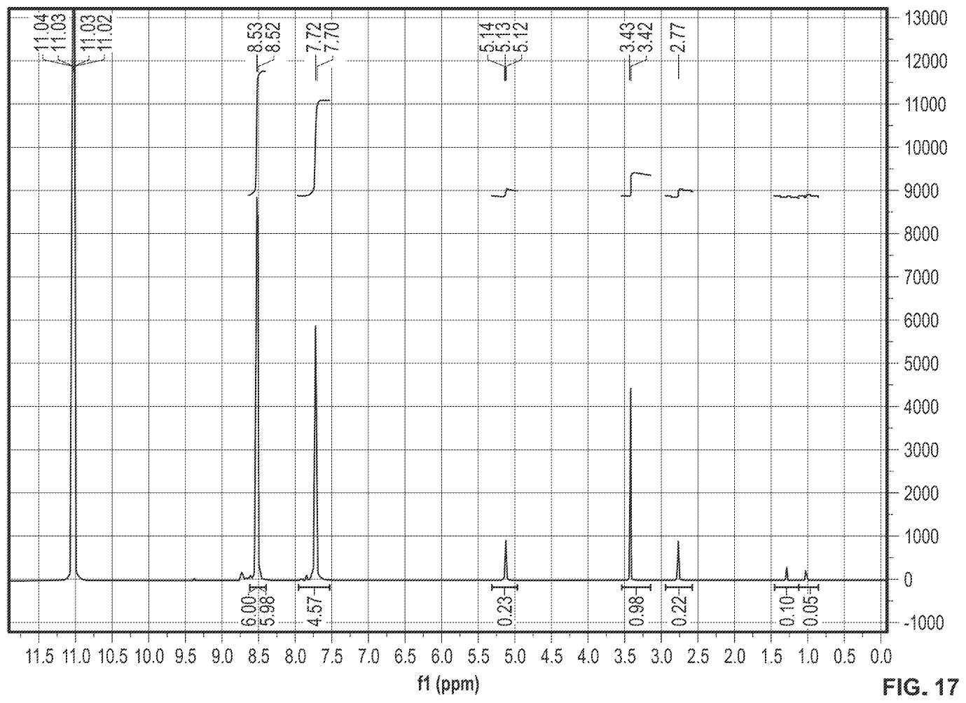

Furthermore, NMR spectroscopy was used to verify the success of the reaction (FIG. 17). In a ¹H-NMR experiment, two new non-aromatic triplet signals showed at 2.8 ppm and 5.2 ppm with identical integration correlations, compared with the spectrum of the precursor.

Moreover, contact angle measurements were carried out on this product as well. They showed a further decrease of the water contact angle of previously 77° of the precursor to 70° of the PEEK-COOH. This result indicates a further increase in hydrophilicity.

### Example 4

### Coupling of Linker Molecules—PEEK-Maleimide

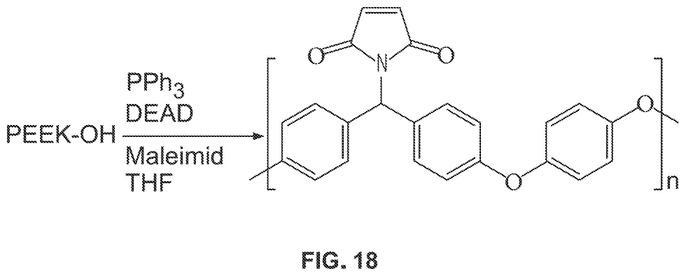

Coupling reagents, which can form bonds with thiol groups, such as maleimide linkers, are widespread among protein and other bioconjugation techniques. In proteins, thiol groups are often involved in disulfide bonds, crosslinking via such groups changing the protein structure only insignificantly. Thiol groups also occur in most proteins, but they are not as numerous as primary amines and make the coupling reaction much more selective. Another advantage of a thioether bond is its irreversibility. The Mitsunobu reaction is often used for bonding such molecules. The reaction scheme of the coupling of the maleimide to PEEK-OH is shown in FIG. 18.

For this purpose, the PEEK-OH sheet was immersed in a solution of triphenylphosphine, diethyl azodicarboxylate, and maleimide in THF and stirred for 24 hours at room temperature. Thereafter, the sheet was washed with a solution of ether/hexane (1:1) four times and subsequently dried in a vacuum oven for at least three hours at 60° C. and 50 mbar. The product PEEK-maleimide will be mentioned hereinafter.

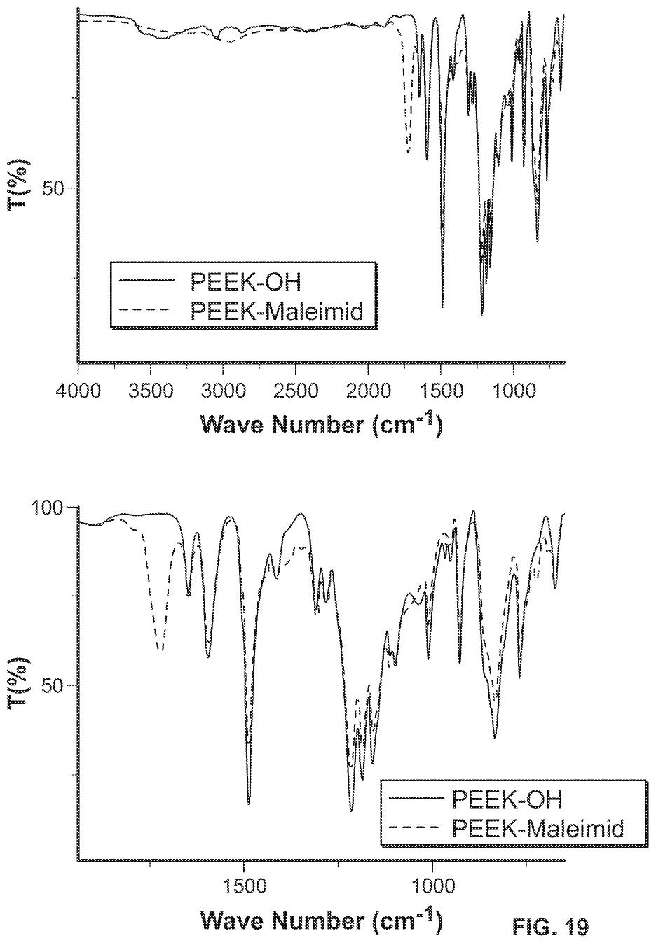

Subsequently, the PEEK-maleimide sheet was examined with ATR-IR spectroscopy. In the spectrum (FIG. 19), the creation of new bands at 2870 cm⁻¹and 2936 cm⁻¹ can be seen, which were assigned to the C—H stretching vibration. Other new signals are at 1789 cm⁻¹ and 1717 cm⁻¹, which were assigned to the symmetric carbonyl stretching vibrations, 1440 cm⁻¹, which was assigned to the symmetrical C—N—C stretching vibration, and at 722 cm⁻¹ and 693 cm⁻¹, which was assigned to ring torsion.

An analysis with energy-dispersive X-ray spectroscopy (EDX) shows the presence of nitrogen on the surface. Under almost neutral reactions conditions (pH 6.5 to 7.5), preferably stable thioether bonds form. Under more alkaline reaction conditions (pH>8.5), preferably primary amines bind, wherein at the same time the hydrolysis rate of the maleimide group to an unreactive maleamic acid increases.

### Example 5

### Coupling of Linker Molecules—PEEK NCO

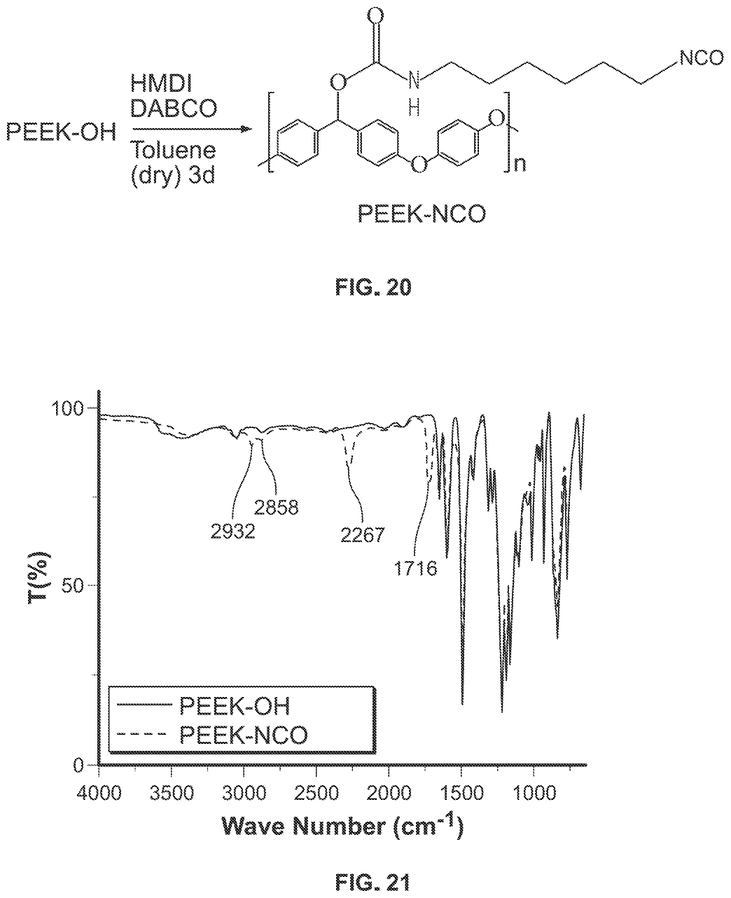

In a parallel approach, a homobifunctional hexamethylene diisocyanate (HMDI linker) was used. It can form stable isourea bonds with amines, among others. Such isocyanate linkers are also capable of coupling molecules including a hydroxyl group, such as polysaccharides, in a carbamate/urethane bond. For the modification of PEEK, the PEEK-OH sheet was immersed in a solution of hexamethylene diisocyanate and catalytic amounts of 1,4-diazabicyclo[2.2.2]octane (DABCO) in toluene and stirred for three days at room temperature. The reaction was carried out in a protective atmosphere under dry reaction conditions. After the reaction time, the sheet was washed twice with toluene and with acetone and finally dried in a vacuum oven for at least three hours at 30° C. and 50 mbar. The resulting product PEEK-NCO will be mentioned hereinafter. The reaction scheme of the coupling of hexamethylene diisocyanate to PEEK-OH is shown in FIG. 20.

After the reaction, the PEEK-NCO sheets were examined with ATR-IR spectroscopy. The spectrum (FIG. 21) shows a new band at 2267 cm⁻¹, which was assigned to the isocyanate group, two additional bands at 2932⁻¹ and 2858 cm⁻¹, which were assigned to the CH stretching vibration of the linker, and a band at 1716 cm⁻¹, which was assigned to the carbonyl stretching vibration of the amide. The two bands observed at about 3420 cm⁻¹ land about 3348 cm⁻¹ probably result from a partial reduction of the isocyanate group owing to the presence of humidity.

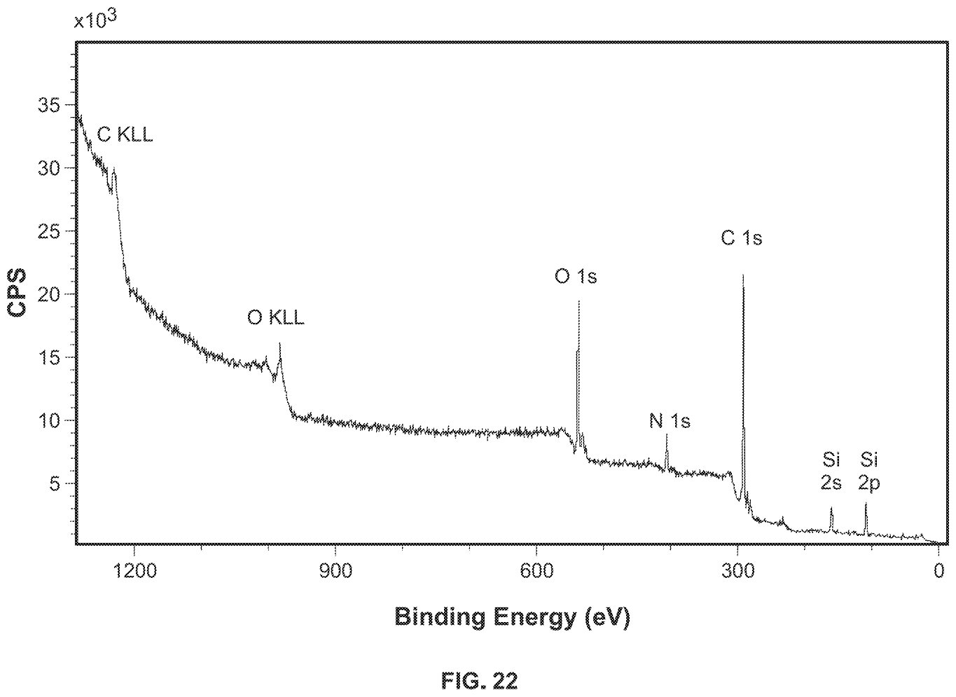

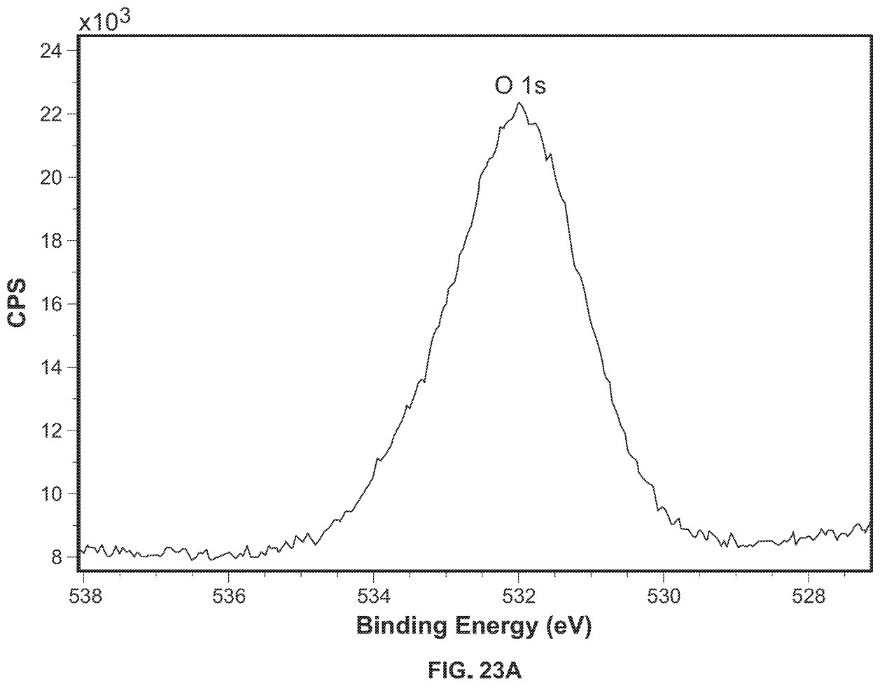

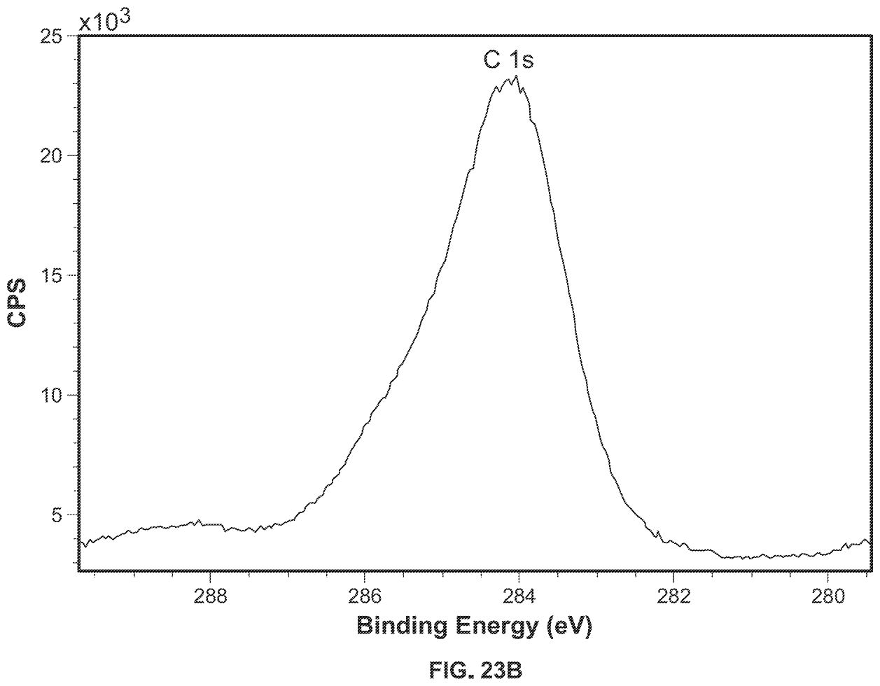

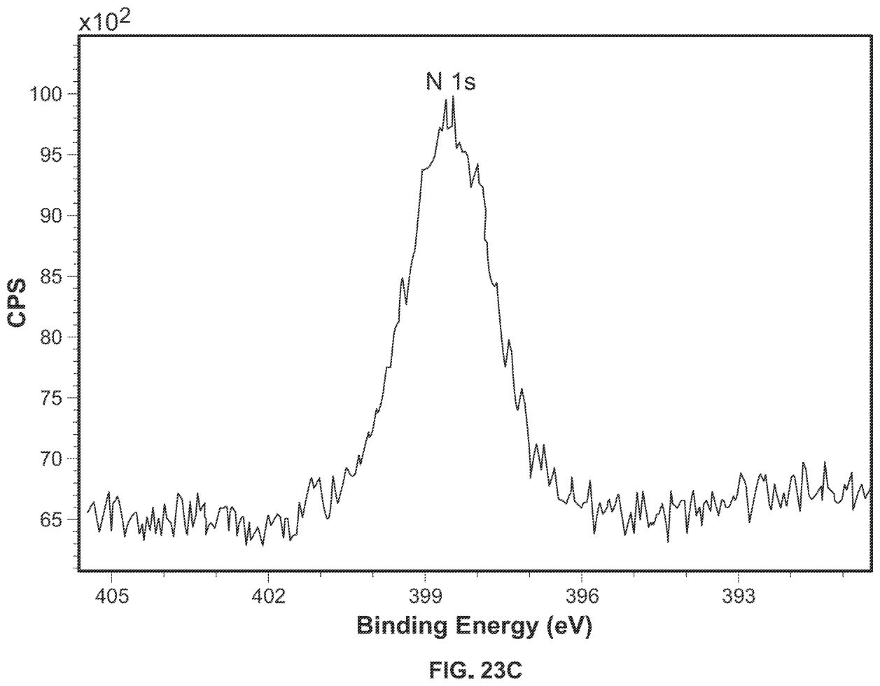

The surfaces of the material were further examined with XPS and compared to the unmodified material. The reference material used here was silicon. The overview spectrum is shown in FIG. 22. It shows a signal at 399.1 eV and thus shows the presence of nitrogen at the surface. The fine spectra of the C_{1S}, O_{1S} and Nis components are shown in FIGS. 23A-23C. The results of the fits are summarized in Table 3. The fine structure of the Nis component shows the presence of nitrogen in two different chemical environments at 399.9 eV and 399.0 eV with relative intensity of 30% to 70%. A quantitative analysis showed a total nitrogen content of 9.2% in the analysis volume. The carbon content was 54.92%. Individual chemical environments of the carbons were identified by fitting the fine spectrum of the C_{1S} signal. They showed signals at 284.7 eV, which were assigned to the C—C/C—H component, 285.2 eV, which were assigned to the CH₂ component, 287.0 eV, which were assigned to the C═O component of the amide, and 289.3 eV, which were assigned to the C═O component of the NCO. The respective relative concentrations were 39.1%, 53.2%, 1 35%, 2.85%. Here, a further component was identified at 286.5 eV. Whether it resulted from the C—O, C—OH or O═C component (unmodified PEEK) was not determined in detail.

The oxygen content was 35.6% with an O_{1S} component at 532.0 eV. By fitting the fine spectrum, oxygen was identified in three different chemical environments. A signal at 531.1 eV, which was assigned to the C═O component, a signal at 532.6 eV, which was assigned to the O—H component, and a signal at 533.3 eV, which was assigned to the O—C component. These signals have a relative intensity of 22.6%, 9.9%, and 57.5%. In this evaluation, however, it has to be taken into account that the HMDI linker can undergo many side reactions, which with this type of evaluation can have negative effects. For example, HMDI linkers among each other can form a dimer or multimer on the surface thus forming an allophanate group. As only the top few nanometers are analyzed through XPS, it is difficult to estimate the exact component concentration after such polymerization of the linkers.

TABLE 3
Summary of XPS measurement of PEEK-NCO.

| | | | Component | Element |
|---|---|---|---|---|
| | Element/ | BE | composition | concentration |
| Assignment | Transition | [eV] | [at. %] | [at. %] |
| Carbon | C 1s | 284.7 | 39.1 | 54.92 |
| | C 1s | 285.2 | 53.2 | |
| | C 1s | 287.0 | 1.35 | |
| | C 1s | 289.3 | 2.85 | |
| | C 1s | 285.5 | 2.4 | |
| | C 1s | 286.5 | 1.06 | |
| Oxygen | O 1s | 531.1 | 22.6 | 35.6 |
| | O 1s | 532.6 | 19.9 | |
| | O 1s | 533.3 | 57.5 | |
| Nitrogen | N 1s | 399.9 | 30 | 9.2 |
| | N 1s | 399.0 | 70 | |
| Silicon | Si 2s | 149 | | 0.28 |

Due to lack of hydrolysis resistance of the HMDI linkers to water, a contact angle measurement was dispensed with.

Scanning electron microscopy shows a relatively smooth, homogeneous surface. An analysis with EDX confirmed the presence of nitrogen on the surface. The nitrogen concentration was 7.67% here.

### Example 6

### Coupling of Biomolecules—Coupling Via EDC

To test the reaction process and for the optimization of reaction conditions, the gelatin was selected as to be biomolecule to be coupled in view of the higher costs of collagen, as gelatin is mainly denatured collagen.

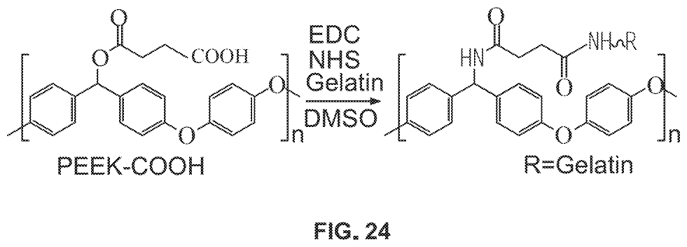

Coupling of the gelatin was carried out with the aid of 1-ethyl-3-(3-dimethylamino-propyl)carbodiimide hydrochloride (EDC). This is a widely used and commercially available coupling reagent, which is frequently used for the chemical coupling of proteins and peptides, proteins and oligonucleotides or biomolecule and particle surfaces. Together with N-hydroxysuccinimide (NHS), a reaction of carboxylates and amines thus forming an amide bond is promoted in particular. EDC coupling reactions are usually carried out under acidic reaction conditions (pH 4.5 to 5.5). The reaction scheme of the coupling of gelatin to PEEK-COOH is shown in FIG. 24.

In order to prevent easy coupling of the gelatin molecules among each other, first the surface of PEEK-COOH was activated with the EDC/NHS solution, and the films were finally transferred from this solution into a gelatin solution and stirred for a further four hours. Subsequently, the sheet was washed at least three times with 40° C.-warm water to wash off adsorbed gelatin. Finally, the sheet was washed with ethanol and dried, which at the same time precipitates the remaining gelatin molecules on the surface. The resulting sheet will be referred to as PEEK gelatin hereinafter.

The PEEK gelatin sheets were finally examined with ATR-IR spectroscopy. Here, however, no significant differences in the spectrum compared to the precursor were detected. This is probably due to the overlap of the very strong characteristic bands for the PEEK material and the comparatively weaker bands of amide bands, which would also have to appear in the same wave number range

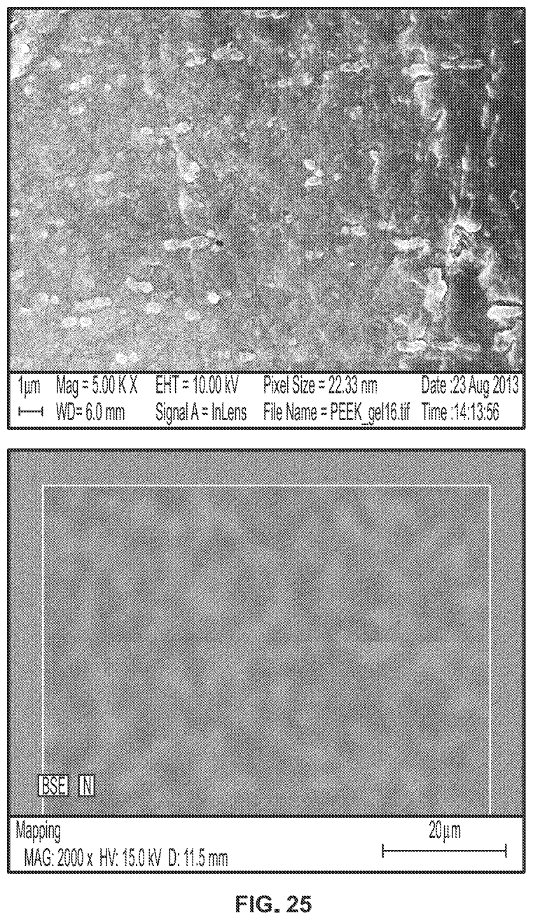

To verify the success of the reaction of the reaction nonetheless, further analysis methods were used. The surface was further examined with scanning electron microscopy. The image (FIG. 25) shows homogeneously distributed, disordered structures on the surface of the sheet, which were not found on the unmodified material or the precursor. Since the film has previously been washed with warm water several times, no adsorbed gelatin should be present at the surface and all visible structures on the surface should be covalently bound gelatin. A directly subsequent EDX analysis shows the presence of nitrogen on the surface. The nitrogen can in this case only originate from the coupled gelatin.

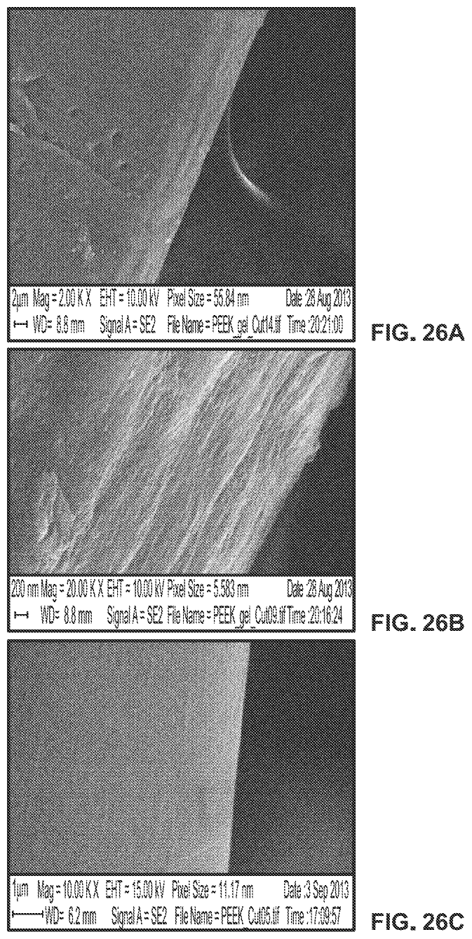

To get an impression of the thickness of the gelatin layer, the gelatin PEEK sheet was cut and the edge face was examined by means of scanning electron microscopy (FIGS. 26A and 26B). This examination showed that the gelatin layer seems to be 100 to 150 nm thick on average.

A thermogravimetric examination revealed a mass fraction of 25 percent by weight of gelatin in the analysis substance.

### Example 7

### Coupling of the Biomolecules—Coupling Via Isocyanate Linker

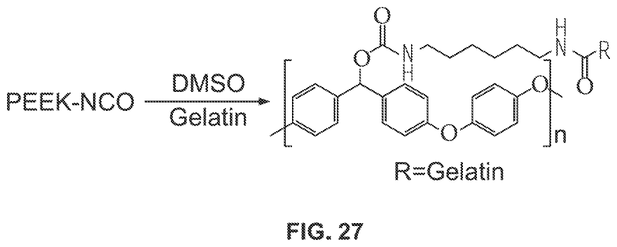

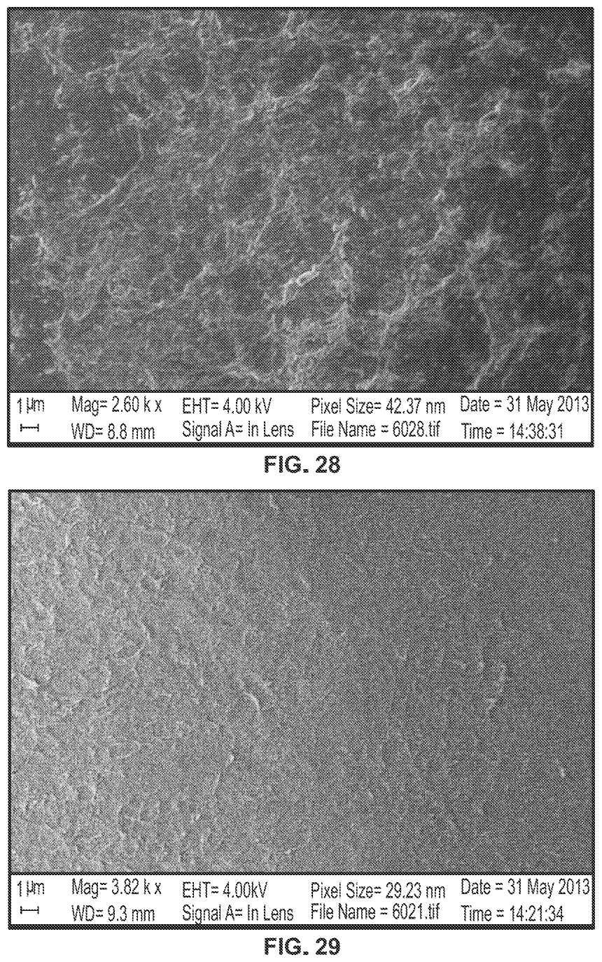

Coupling of the gelatin to the surface of the PEEK-NCO took place from a solution of gelatin in DMSO. The reaction scheme of the coupling of gelatin via an isocyanate linker is shown in FIG. 27. Also in this case was the surface examined with scanning electron microscopy. The image (FIG. 28) shows homogeneously distributed, disordered structures on the surface of the sheet, which were not found on the unmodified material or the precursor. Since the sheet has previously been washed with warm water several times, no adsorbed gelatin should be located at the surface and all visible structures on the surface should be covalently bound gelatin. A complementary EDX analysis was not used for verification in this case, since there is nitrogen at the surface only due to the isocyanate linker.

ATR-IR spectroscopic examinations showed a disappearance of the characteristic isocyanate signal at 2267 cm′ in a before/after comparison, which is indicative of a gelatin coupling having taken place.

### Example 8

### Coupling of Biomolecules—Coupling Via Maleimide Linker

Coupling of the gelatin to the surface of the PEEK maleimide took place from a solution of gelatin in water. For this purpose, the PEEK maleimide sheet was stirred in a 3% solution of gelatin in water for 3 hours at room temperature. Subsequently, the sheet was washed three times with warm water, and finally was washed once with acetone. Drying of the sheet was carried out in a vacuum oven for at least three hours at 40° C. and 50 mbar. Also in this case was the surface examined with scanning electron microscopy. The image (FIG. 29) shows disordered structures on the surface of the sheet, which were not found on the unmodified material or the precursor. Since the film has previously been washed with warm water several times, no adsorbed gelatin should be located at the surface and all visible structures on the surface should be covalently bound gelatin. Again, a complementary EDX analysis was not used for verification here, since there is nitrogen at the surface only due to the maleimide linker.

ATR-IR spectroscopic examinations showed a disappearance of the characteristic isocyanate signal at 2870 cm¹ in a before/after comparison, which is indicative of a gelatin coupling having taken place.

### Example 9

### Biomineralisation

To finally mineralize the covalently coupled gelatin layer, the PEEK gelatin sheet was immersed in a 2-(4-(2-hydroxyethyl)-1-piperazinyl)ethanesulfonic acid (HEPES) buffer solution containing calcium chloride, dipotassium hydrogen phosphate, and poly-aspartic acid for four days at 37° C.

Finally, the sheets were washed with water at least three times and with ethanol three times. Drying of the material was carried out in a vacuum oven (40° C. at 50 mbar). The resulting material will be referred to as PEEK-HAp hereinafter.

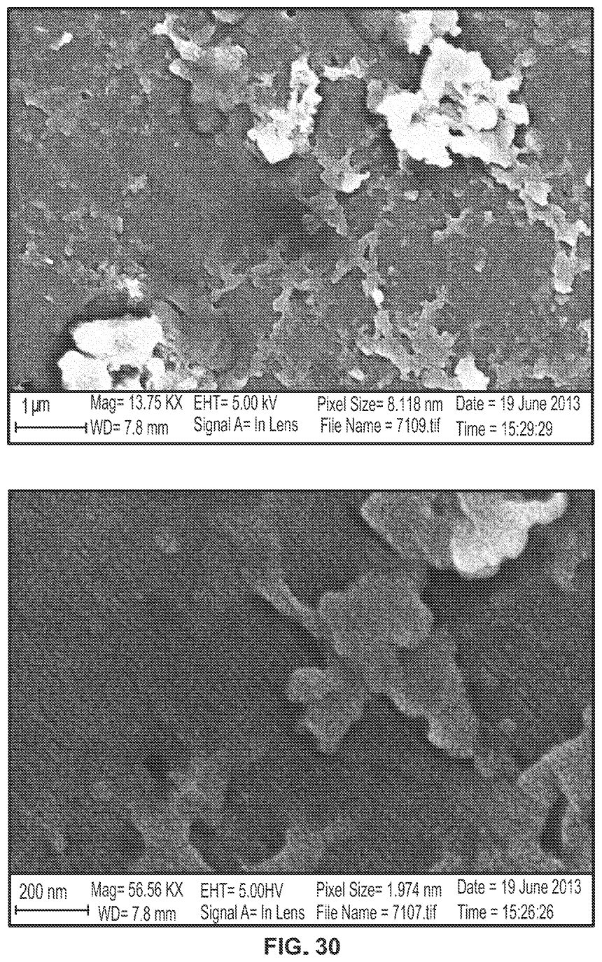

The surface was examined using scanning electron microscopy (FIG. 30). Here, some large and randomly distributed structures were identified on the surface, which seem to grow out of the surface. Between these large structures, smaller structures with a structure similar to the bigger ones were identified. A directly subsequent EDX analysis showed that these structures, as well as the entire surface, contained calcium and phosphorus components. The results are summarized in Table 4. Since the surface has a greater roughness, these results are of a more qualitative nature and confirm that the detected structures and the surface are composed of calcium phosphate. For quantification by means of EDX, the surface must be polished to be smooth, or another method, such as thermogravimetry, must be used.

TABLE 4
Summary of the EDX results of the mineralized PEEK sheet

| | | | Mass percent | Atomic |
|---|---|---|---|---|
| | | | (norm) | percent |
| Element | AN | Seriess | [wt %] | [at. %] |
| Carbon | 6 | K-Series | 77.48 | 82.45 |
| Oxygen | 8 | K-Series | 21.52 | 17.19 |
| Calcium | 20 | K-Series | 0.64 | 0.2 |
| Phosphorus | 15 | K-Series | 0.23 | 0.1 |
| Sulfur | 16 | K-Series | 0.14 | 0.05 |

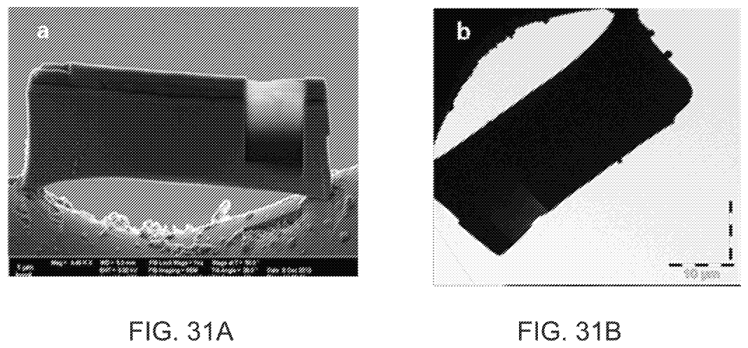

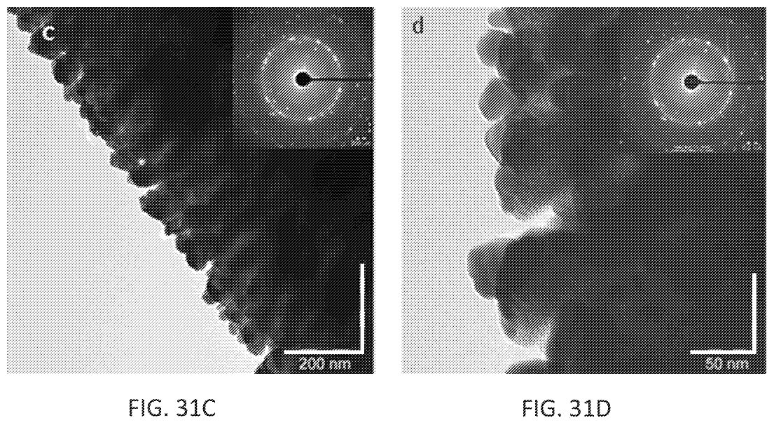

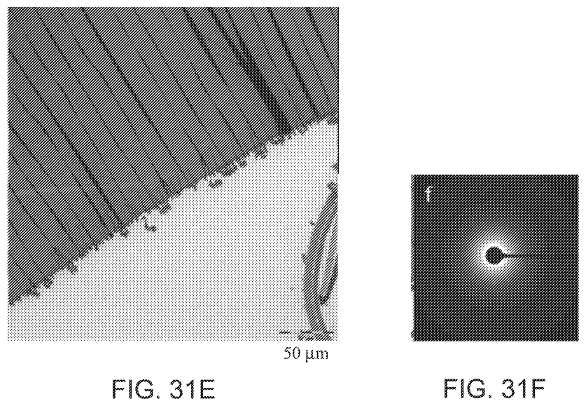

To identify this calcium phosphate layer, a thin cut through the material was prepared by means of an FIB cut (FIGS. 31A-31D) or ultramicrotome cut (FIG. 31E) and finally examined with transmission electron microscopy. A thin layer, to which presumably the gelatin/apatite surface modification corresponds, can be recognized directly on the surface of the PEEK material. Examination of these structures by electron diffraction showed that the crystalline phase of this layer presumably consists of amorphous calcium phosphate. The crystalline signals of the diffraction images in FIGS. 31C and 31D could clearly be assigned to gold, which had to be applied to the sample as a conductor material for scanning electron microscopy and the FIB cut. No further crystalline signals were taken from these diffraction images in this example, which confirmed the assumption of an amorphous mineral phase. From diffraction images of photographs of ultramicrotome cuts of the material (FIGS. 31E, 31F), the amorphous character of the mineral phase was confirmed. Also in the TEM images of the microtome cuts is the surface modification of the PEEK material with a gelatin/calcium phosphate layer clearly visible. The stripes in the material itself (FIG. 31E, top left) result from the unfolding of the very thin cut of the material during the cutting process with the ultramicrotome.

### Example 10

### In Vitro Evaluation of the Materials

In order to examine the biocompatibility, the PEEK-HAp sheets were finally subjected to various in vitro tests. These tests were conducted according to the EN ISO 10993-5:2009 directive on the biological evaluation of medical devices, in particular part 5 for assessing the in vitro cytotoxicity of biomaterials. All tests were carried out in appropriate statistical replicas.

First, by means of an indirect toxicological study, it was intended to find out of whether toxic substances can be detached from or dissolved out of the material. For testing the extracts, the material 24 was swirled in cell culture medium at 37° C. for 24 hours. Finally, the cell culture medium was incubated in cell culture medium with L-929 fibroblasts (P14) for two days in undiluted form and at dilutions of 1:2, 1:4, and 1:10. Subsequently, the degree of cell destruction was analyzed by means of light microscopy, and cell viability was analyzed by means of an MTT assay. The applicability of this test was confirmed by means of a positive and negative control. Light microscopy showed that no more than 10% of the cells on PEEK-gelatin films and PEEK-HAp films have a round shape and that no discrete intracellular granules were in the cells, which corresponds to non-reactivity overall. Also, no cell lysis or blank areas were found. The MTT test showed a cell viability of over 90%. Thus, the materials can be considered to be non-toxic.

Complementarily, a direct toxicological examination was performed on PEEK and PEEK-HAp material. To this end, NIH 3T3 fibroblasts were incubated directly on the material for 24 hours and, thereafter, the relative cell viability using an MTT assay was determined. A light-microscopic evaluation was carried out only partly due to the opacity of the material. The applicability of this test was also confirmed by application of a positive control. With the light-microscopic examination, no cell lysis or blank areas were found. The MTT test showed a cell viability of the cells of over 95%±0.2.9. The material can thus be regarded as non-toxic.

After the toxicological studies, the relative proliferation, viability, and adhesion behavior of human fibroblasts (NHDF—“normal human dermal fibroblast”) of a low passage number on the modified material as compared to the unmodified material was examined.

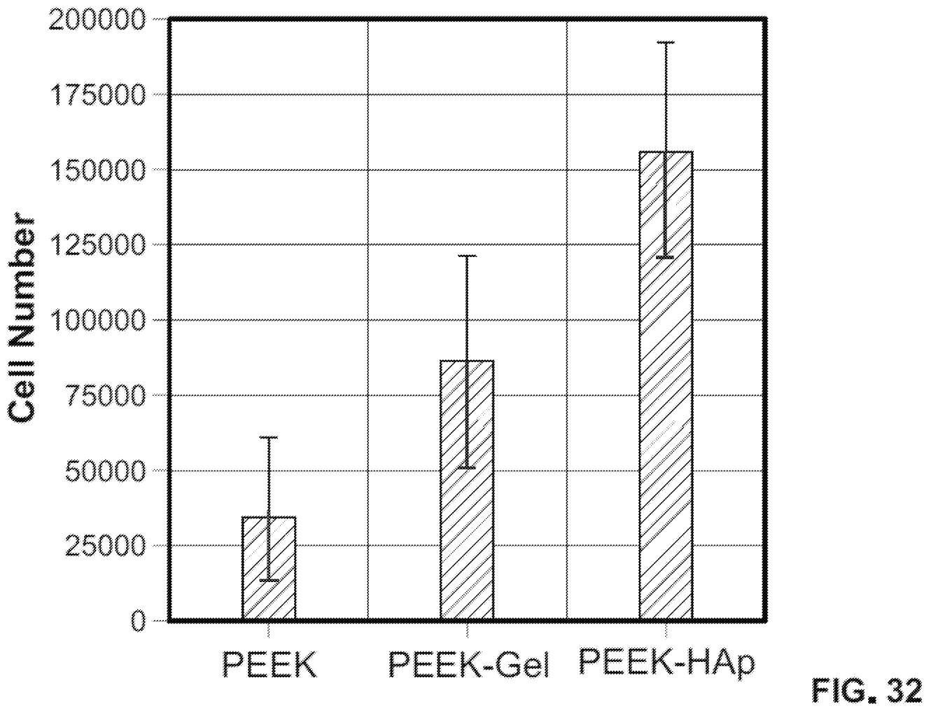

For proliferation, a certain number of cells was incubated on the surface of the materials for 24 hours. Thereafter, the cells were released from the material and their number was determined using a CASY counting device. This examination showed that the cells proliferate better on the PEEK-HAp material by a factor of 4.4 than on the unmodified PEEK. At the same time, the proliferation was examined at the intermediate stage of the PEEK-gelatin material. It was found that the fibroblasts proliferate better by a factor of 2.5 than on the unmodified material. Thus, it follows that the fibroblasts proliferate better on the PEEK-gelatin material and even better on the PEEK-HAp material. The results are shown in FIG. 32.

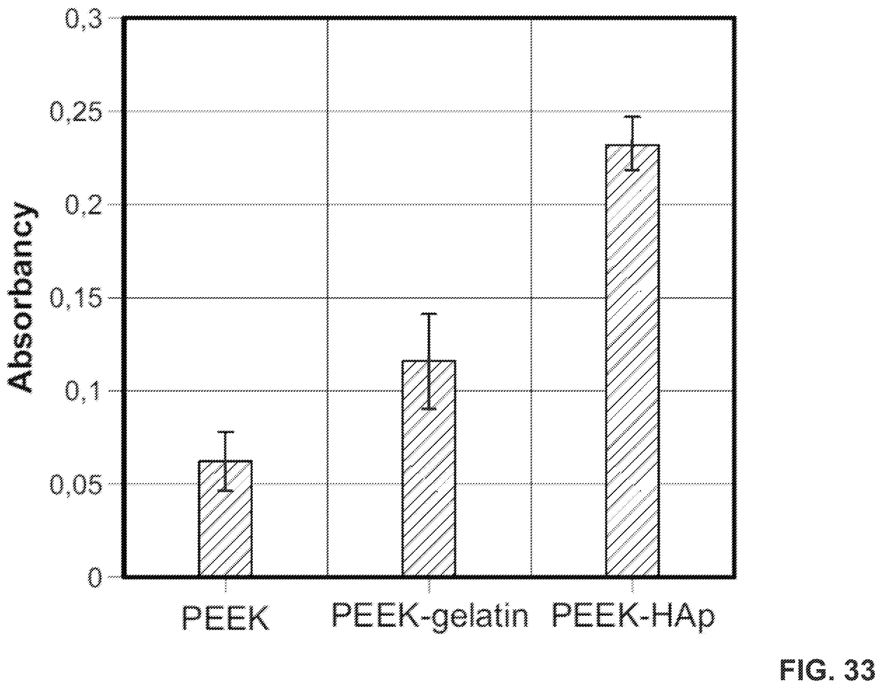

Complementarily, the cell viability was examined using an MTT assay. This examination showed that the cell viability on the PEEK-gelatin material is higher by a factor of 1.8, based on the unmodified PEEK material, and on the PEEK-HAp material higher by a factor of 3.9. Since the cell viability is directly correlated with the cells number, this test is also a control for the proliferation test. This test also shows that the fibroblasts proliferate better on the PEEK-gelatin material and even better on the PEEK HAp material than on the unmodified PEEK. The results are shown in FIG. 33.

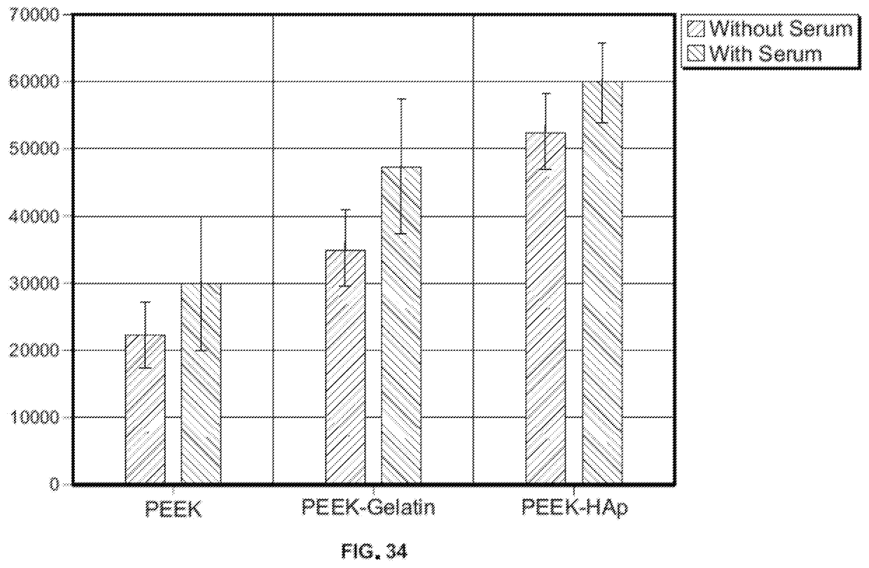

The cell adhesion of the fibroblasts on the materials was also examined. For this, a certain number of cells was seeded on the materials and incubated on the material for an adhesion time typical of fibroblasts. After this time, all non-adherent cells were washed off. In this step, a reproducible wash rate is particularly important. After this washing step, the adherent cells were detached from material and their number was determined by means of a Neubauer counting chamber. This examination showed that the fibroblasts, in the absence of cell culture serum, adhere better on the PEEK-gelatin material by a factor of 1.5 and on the PEEK-HAp material by a factor of 2.3 than on the unmodified PEEK. This assay was performed both in the presence and in the absence of serum in cell culture medium. This is based on the basis that it has to be examined whether the altered adhesion of the cells is due to the proteins in the cell culture medium, which adsorb on the material surface, or due to the surface modification made. The presence of cell culture serum increases the adhesion of fibroblasts by 28% on average. The results are shown in FIG. 34.

Other features which are considered as characteristic for the invention are set forth in the appended claims.

Although the invention is illustrated and described herein as embodied in material for bone implants and method of producing same, it is nevertheless not intended to be limited to the details shown, since various modifications and structural changes may be made therein without departing from the spirit of the invention and within the scope and range of equivalents of the claims.

1. A method for producing a material for bone implants, comprising the steps of:
(a) providing a surface comprising a material selected from the group consisting of oxidic ceramic materials and polyether ether ketone (PEEK),
(b) covalently coupling a matrix, comprising collagen, gelatin, or combinations thereof, to the surface, and
(c) mineralizing the matrix with calcium phosphate,
wherein the surface comprises polyether ether ketone (PEEK) and the collagen, gelatin, or combinations thereof is bound to the PEEK by a linker selected from the group consisting of a dicarboxylic acid linker, a maleimide linker, and a hexamethylene diisocyanate linker.

2. The method according to claim 1, wherein the surface comprises polyether ether ketone (PEEK).

3. The method according to claim 1, wherein the material for bone implants comprises:
(a) a surface comprising a material selected from the group consisting of oxidic ceramic materials and polyether ether ketone (PEEK),
(b) a matrix covalently bound to the surface, the matrix comprising collagen gelatin, or combinations thereof, and
(c) a calcium phosphate embedded into the matrix.
